(19) United States
(12) Patent Application Publication
(10) Pub. No.: US 2002/0035317 A1
(43) Pub. Date: Mar. 21, 2002

(54) Optical imaging system with movable scanning unit

(75) Inventors: Xuefeng Cheng, Milpitas, CA (US); Xiaorong Xu, Menlo Park, CA (US); Shuoming Zhou, Cupertino, CA (US); Lai Wang, Cupertino, CA (US); Ming Wang, San Jose, CA (US); Feng Li, San Jose, CA (US); Guobao Hu, San Jose, CA (US)

Correspondence Address: Pennie & Edmonds, LLP, 3300 Hillview Avenue, Palo Alto, CA, 94304, US

(73) Assignee: Photonify Technologies

(21) Appl. No.: 09/778,613

(22) Filed: Feb. 6, 2001

Related U.S. Application Data
(60) Provisional application No. 60/223,074, filed on Aug. 4, 2000.

(51) Int. Cl. A61B 5/00

(52) U.S. Cl. 600/322; 600/473; 600/476

(57) ABSTRACT

The present invention generally relates to optical imaging systems and methods thereof for providing images of two- and/or three-dimensional distribution of properties of chromophores in various physiological media. In particular, the present invention provides preferred embodiments of optical imaging systems, optical probes, sensor assemblies, and methods thereof for utilizing movable scanning units. A typical optical imaging system includes at least one wave source, wave detector, movable member, and actuator member. The wave source emits electromagnetic waves into a target area of the physiological medium and the wave detector generates output signal in response to electromagnetic waves detected thereby. The wave source and detector are disposed at the movable member which is moved by the actuator member so that at least one of the wave source and detector moves over different regions of the target area while generating the output signal thereby. Accordingly, the optical imaging system and optical probes of the present invention can scan the target area which is substantially larger than the scanning area of its scanning unit.

### CROSS-REFERENCE TO RELATED APPLICATIONS

This application claims the benefit of the filing date of U.S. Provisional Patent Application bearing Ser. No. 60/223,074, entitled “A Self-Calibrated Optical Scanner for Diffuse Optical Imaging” and filed on Aug. 4, 2000.

### FIELD OF THE INVENTION

The present invention generally relates to an optical imaging system capable of providing images of spatial or temporal distribution of chromophores or their properties in a physiological medium. More particularly, the present invention relates to an optical imaging system and optical probe thereof equipped with a movable sensor assembly. The present invention is applicable to optical imaging systems and optical probes thereof whose operation is based on wave equations such as the Beer-Lambert equation, modified Beer-Lambert equation, photon diffusion equation, and their equivalents.

### BACKGROUND OF THE INVENTION

Near-infrared spectroscopy has been used to measure various physiological properties in animal and human subjects. The basic principle underlying the near-infrared spectroscopy is that physiological media such as tissues and cells include a variety of light-absorbing and/or light-scattering chromophores which can interact with electromagnetic waves transmitted thereto and traveling therethrough. For example, human tissues include numerous chromophores among which deoxygenated and oxygenated hemoglobins are the most dominant chromophores in the spectrum range of 600 nm to 900 nm. Therefore, the near-infrared spectroscope has been applied to measure oxygen levels in the physiological media in terms of tissue hemoglobin oxygen saturation (“oxygen saturation” hereinafter). Technical background for the near-infrared spectroscopy and diffuse optical imaging has been discussed in, e.g., Neuman, M. R., “Pulse Oximetry: Physical Principles, Technical Realization and Present Limitations,” Adv. Exp. Med. Biol., vol. 220, p.135-144, 1987, and Severinghaus, J. W., “History and Recent Developments in Pulse Oximetry,” Scan. J. Clin. and Lab. Investigations, vol. 53, p.105-111, 1993.

Various techniques have been developed for the near-infrared spectroscopy, including time-resolved spectroscopy (TRS), phase modulation spectroscopy (PMS), and continuous wave spectroscopy (CWS). In a homogeneous, semi-infinite model, the TRS and PMS are generally used to solve the photon diffusion equation, to obtain the spectra of absorption coefficients and reduced scattering coefficients of the physiological media, and to estimate the concentrations of oxygenated and deoxygenated hemoglobins and oxygen saturation. The CWS has generally been used to solve the modified Beer-Lambert equation and to calculate changes in the concentrations of the oxygenated as well as deoxygenated hemoglobin.

Despite their capability of providing hemoglobin concentrations as well as the oxygen saturation, the major disadvantage of the TRS and PMS is that the equipment has to be bulky and, therefore, expensive. The CWS may be manufactured at a lower cost but is generally limited in its utility, for it can estimate only the changes in the hemoglobin concentrations but not the absolute values thereof. Accordingly, the CWS cannot provide the oxygen saturation. The prior art technology also requires a priori calibration of optical probes before their clinical application by, e.g., measuring a baseline in a reference medium or in a homogeneous portion of the medium. Furthermore, all prior art technology requires complicated image reconstruction algorithms to generate images of two-dimensional and/or three-dimensional distribution of the chromophore properties.

Accordingly, there exist needs for efficient, compact, and relatively cheap optical imaging systems with optical probes capable of measuring the absolute values of the chromophores or their properties and capable of scanning a large target area of the medium in a single measurement.

### SUMMARY OF THE INVENTION

The present invention generally relates to optical imaging systems, optical probes, and methods for providing two- and/or three-dimensional images of spatial and/or temporal distribution of the chromophores or their properties in a physiological medium. More particularly, the present invention relates to optical imaging systems, optical probes, optical sensor assembly, and methods for utilizing movable scanning units (i.e., source-detector arrangements).

In one aspect of the invention, an optical imaging system is provided to generate images of a target area of a physiological medium, where the images generally represent distribution of the chromophores or their properties in the target area of the medium. An exemplary optical imaging system includes at least one wave source arranged to irradiate electromagnetic waves into the medium and at least one wave detector arranged to detect such electromagnetic waves and to generate output signal in response thereto. The optical imaging system also includes a movable member and an actuator member, where the movable member defines a longitudinal axis and includes at least one of the foregoing wave source and detector. The actuator member operationally couples with the movable member and generates at least one movement of the movable member with respect to the target area along at least one curvilinear path.

The optical imaging systems, optical probes thereof, and methods therefor (collectively referred to as “optical imaging system” or “optical probe” hereinafter) of the present invention provides numerous benefits over the prior art optical imaging technology. Contrary to the conventional optical imaging devices which allow a single measurement at each measurement location, the optical imaging system of the present invention provides a scanning unit or source-detector arrangement ( collectively referred to as “scanning unit” hereinafter) which may be positioned in one region of a much larger target area and may be moved through other measurement regions of the target area without having to move other parts of the optical imaging system theretoward. Therefore, per measurement in each target area, the foregoing optical imaging system can scan the target area which is larger than the scanning area of the scanning unit. The optical imaging system of the present invention also requires fewer wave sources and/or detectors than its conventional counterparts. Therefore, such optical imaging system can be constructed as a light and compact article which can be portably worn by a test subject. The optical imaging system of the present invention can reduce or minimize noises attributed to idiosyncratic variances inherent in each of the wave sources and detectors. As a result, the foregoing optical imaging system provides images with improved accuracy and higher resolution. The optical imaging system of the present invention is generally operative regardless of image processing schemes and procedures. Therefore, such optical imaging system may be applied to or may be adopted by any optical imaging devices whose operation is based on wave equations such as the Beer-Lambert equation, modified Beer-Lambert equation, photon diffusion equation, and their equivalents (collectively referred to as “wave equations” hereinafter). The optical imaging system of the present invention also ensures appropriate optical coupling to be maintained between the medium and movable wave sources and/or detectors during the movement of the movable member. Accordingly, a single baseline of the output signal may be obtained and applied throughout the entire target area for calibrating the output signals. In addition, such optical imaging system can provide real-time images of distribution of the chromophores or their properties by employing much simpler and more efficient image construction schemes.

Embodiments of this aspect of the present invention may include one or more of the following features.

Preferably, the wave sources irradiate into the target areas of the medium electromagnetic waves which have different wave characteristics, and the wave detectors detect at least a portion of the foregoing electromagnetic waves emanating from the target area after such waves are absorbed and/or scattered by the chromophores in the medium. Examples of such electromagnetic waves may include, but not limited to, sound waves, near-infrared rays, infrared rays, visible light rays, ultraviolet rays, lasers, and photons.

The wave sources and detectors are generally aligned along the longitudinal axis of the movable member and form at least one scanning unit which is elongated along the same axis and which defines therearound a scanning area in which the wave detector detects the electromagnetic waves emanating from the target area. The scanning area of the scanning unit is generally smaller than the target area.

The movable member includes at least two wave detectors (or sources) that are disposed substantially linearly along its longitudinal axis and interposed between the wave sources (or detectors). Alternatively, one wave source (or detector) may be disposed on one side across the longitudinal axis of the movable member, while another wave source (or detector) disposed on the other side thereacross. Such wave sources (or detectors) may be arranged substantially symmetrically with respect to the longitudinal axis of the movable member.

The actuator member generates movement of the movable member such as, e.g., curvilinear translation, rotation, revolution, reciprocation, and a combination thereof. The actuator member may generate such movement at a constant speed or at speeds varying over time and/or location over the target area. The actuator member may further impart the foregoing movement with temporal characteristics, e.g., an impulse, pulse, pulse train, step, composite steps, sinusoid, and a combination thereof. Such movement may be continuous, intermittent, and/or periodic. The curvilinear path of the movement may be arranged so that at least a portion thereof is orthogonal to, is parallel with or forms an angle with respect to the longitudinal axis of the movable member. The actuator member may generate at least two movements of the movable member along at least two curvilinear paths sequentially. Alternatively, the actuator member may generate at least a portion of one movement and at least a portion of the other movement simultaneously. The actuator member may also be arranged so that at least a portion of one curvilinear path is substantially orthogonal to at least a portion of the other curvilinear path. Such curvilinear paths may be the orthogonal axes of the Cartesian, cylindrical or spherical coordinate systems.

The actuator member may generate a first and second movements of the movable member sequentially, where the first movement starts from a first region of the target area toward a second region thereof and where the second movement starts from the second region toward the first region of the target area. Alternatively, the actuator member may sequentially generate a first movement of the movable member starting from a first side of the target area toward a second side thereof, a second movement of the movable member from the second side to a third side of the target area, and a third movement of the movable member from the third side toward a fourth side of the target area, where the fourth side may be either the first side or second side thereof. Such first and third movements may be substantially curvilinear translations, while the second movement may be substantially a rotation. When the target area has a shape of a rectangle, the first and second sides may be a first pair of opposing sides of the rectangle, while the third and fourth sides may be a second pair of opposing sides of the rectangle. The actuator member may simultaneously generate a first and second movements of the movable member along a first and second curvilinear paths, respectively. At least a portion of the first curvilinear path may also be substantially orthogonal to at least a portion of the second curvilinear path. In the alternative, both of the first and second movements may substantially be linear reciprocations.

In another aspect of the present invention, an optical imaging system may include at least one sensor assembly having at least one of the foregoing wave sources and at least one of the foregoing wave detectors, a body arranged to mechanically support at least a portion of the sensor assembly, and an actuator member operationally coupling with the sensor assembly and/or the body and arranged to generate the movement of the sensor assembly and/or the body with respect to the target area of the medium along at least one curvilinear path.

Embodiments of this aspect of the present invention may include one or more of the following features.

The sensor assembly may fixedly couple with the body so that the actuator member moves both the sensor assembly and body in unison with respect to the target area. Alternatively, the sensor assembly may movably couple with the body so that the actuator member may generate a first movement of the sensor assembly with respect to the body and target area and a second movement of the body with respect to the target area. The actuator member may generate at least a portion of the first movement simultaneously with at least a portion of the second movement. Alternatively, the actuator member may generate the first and second movements sequentially. Such sensor assembly and body may be constructed as a hand-held portable probe. In a separate embodiment, the sensor assembly or body may include a moving unit arranged to move both of the sensor assembly and body to different regions of the target area and/or different target areas of the medium.

In yet another aspect of the invention, an optical imaging system includes at least one portable probe and a console. The portable probe generally includes at least one movable member and an actuator member. The movable member includes at least one of the foregoing wave sources and at least one of the foregoing wave detectors. The actuator member operationally couples with the movable member and generates movement of the movable member along at least one curvilinear path. The console includes an imaging member which receives the output signal, determines the distribution of the chromophores or their properties by obtaining a set of solutions from the wave equations applied to the wave sources and/or detectors, and generates images of the two- and/or three-dimensional distribution of the chromophores or their properties.

The foregoing aspect of the present invention offers several advantages over the prior art counterparts. For example, bulky or heavy components may be incorporated into the console, while only essential elements are included in the portable probe, thereby allowing construction of a compact and light portable probe. Because such portable probe needs fewer components, idiosyncratic component variances and noises attributed thereto may also be minimized. Furthermore, such portable probe can be worn by a test subject for constant or periodic monitoring of the chromophores or their properties in the target area of the medium of the test subject.

Embodiments of this aspect of the present invention may include one or more of the following features.

The foregoing optical imaging system includes a connector member which is arranged to provide, e.g., electrical signal or data communication, optical signal or data communication, electric power transmission, mechanical power transmission, and/or data transmission between the portable probe and the console. More particularly, a fiber optic article such as an optical fiber may be used to provide the optical communication between the portable probe and console. The portable probe can also be made as a separate article which includes its own rechargeable power supply unit. Such probe may be constructed to be detachable from the console and to transmit various signals and/or data to the console telemetrically. Such portable probe may also include a memory member capable of storing the foregoing signals and/or data.

In a farther aspect of the present invention, an optical imaging system may include at least one optical probe having thereon at least one of the foregoing wave sources and at least one of the foregoing wave detectors. The optical probe may include at least one movable member and an actuator member as well. The movable member includes at least one of the wave source and detector, while the actuator member operationally couples with the movable member and generates movement of the movable member along at least one curvilinear path. The optical imaging system also includes a console which operationally couples with the optical probe and which includes an imaging member arranged to receive the output signal, to determine the distribution of the chromophores or their properties by obtaining a set of solutions from the wave equations applied to the wave sources and/or detectors, and to generate images representing two- and/or three-dimensional distribution of such chromophores or their properties.

In yet another aspect of the invention, an optical imaging system includes at least two of the foregoing wave sources and at least two of the foregoing wave detectors, where at least two wave sources and at least two wave detectors are disposed substantially linearly along a straight line.

Embodiments of this aspect of the present invention may include one or more of the following features.

All of the wave sources and detectors may be disposed substantially linearly along the straight line. An actuator member may generate movement of some or all of the wave sources and/or detectors. The optical imaging system may further include a movable member which in turn includes all of the foregoing wave sources and detectors therein. The actuator member may also generate another movement of the movable member in addition to the movement of the wave sources and/or detectors.

In another aspect, a method is provided for generating images of target areas of a physiological medium by an optical imaging system, where such images are generally the distribution of absolute or relative values of the chromophores or their properties in a physiological medium. The optical imaging system includes at least one of the foregoing wave sources and at least one of the foregoing wave detectors, a movable member, and an actuator member. The movable member has a longitudinal axis, includes at least one of the wave source and detector, and operationally couples with the actuator member. The wave source and detector form a scanning unit which is elongated along the longitudinal axis of the movable member and which defines a scanning area therearound. The actuator member generates at least one movement of the movable member along at least one curvilinear path. The image generating method includes positioning the scanning unit in a first region of the target area, scanning the first region by irradiating electromagnetic waves into the medium by the wave source and by generating the output signal in response to the electromagnetic waves detected by the wave detector, and manipulating the actuator member to generate movement of the movable member starting from the first region to a second region of the target area of the medium along at least one curvilinear path.

Embodiments of this aspect of the present invention may include one or more of the following features.

The image generating method includes determining the distribution of the chromophores or their properties in the first region of the target area and then obtaining the images for the foregoing distribution. The image generating method may include repeating the foregoing scanning and manipulating steps after positioning the scanning unit in at least two or more regions of the target area. The image generating method may further include determining the distribution of the chromophores or their properties in the first region of the target area and obtaining the images for the foregoing distribution in the same first region of the same target area.

The positioning step includes providing optical coupling between the wave sources and the medium and between the wave detectors and the medium, and maintaining such optical coupling during the movement of the movable member.

The manipulating step includes moving the movable member at a constant speed or at speeds which vary with respect to time and/or position over the target area. The actuator member may move the movable member along the curvilinear path which may be substantially orthogonal to, be parallel with or form a pre-selected angle with respect to the longitudinal axis of the movable member. The manipulating step may also include at least one of linearly translating the movable member along a linear path, translating it along at least one curvilinear path, rotating it about a center of rotation around a desired angle along at least one curved path, revolving it about a center of rotation for a desired number of turns along at least one curved path, and/or reciprocating it along the same or different curvilinear paths. The manipulating step may further include generating at least two movements of the movable member along at least two curvilinear paths.

In a further aspect of the invention, a method is provided for generating the images of target areas of a physiological medium by an optical imaging system including a sensor assembly having thereon at least one of the foregoing wave sources and at least one of the foregoing wave detectors, a body mechanically supporting at least a portion of the sensor assembly, and an actuator member operationally coupling with the sensor assembly and/or body and capable of generating at least one movement of the sensor assembly and/or body. The image generating method includes positioning the sensor assembly in a first region of the target area of the medium, scanning the first region with the sensor assembly by irradiating electromagnetic waves thereinto and detecting such waves therefrom, and manipulating the actuator member to generate the movement of the sensor assembly and/or body starting from the first region toward an adjacent second region of the target area along at least one curvilinear path.

Embodiments of this aspect of the present invention may include one or more of the following features.

The image generating method includes fixedly coupling the sensor assembly with the body, thereby rendering the sensor assembly move with the body in unison during the movement of the sensor assembly. Alternatively, the image generating method includes movably coupling the sensor assembly with the body and moving the sensor assembly with respect to at least one of the body and the target area during the foregoing movement. The actuator member may generate another movement of the body so that the sensor assembly may be moved to and repositioned in different target areas sequentially or simultaneously with the movement of the body.

In a further aspect, a method is provided for generating the images of target areas of a physiological medium by an optical imaging system including multiple wave sources and wave. The image generating method includes substantially linearly disposing at least two wave sources (detectors) along a straight line in a region of the target area, and aligning at least two wave detectors (or sources) in the region of the target area so that at least two wave detectors (or sources) are disposed substantially linearly along the straight line. Accordingly, the scanning unit is defined around the wave sources and detectors, and forms an elongated scanning area which is smaller than the target area.

Embodiments of this aspect of the present invention may include one or more of the following features.

The image generating method may include scanning one region of the target area and generating at least one movement of the wave sources and detectors so as to move these sensors (such as the wave sources and detectors) to another region of the target area. In addition, the scanning and generating steps may be repeated in different regions of the target area so that the optical imaging system can scan different regions of the target area all of which have a total area substantially greater than the scanning area of the scanning unit or which amounts to a desired fraction of the target area. The foregoing repeating step may be terminated after a desired number of repetitions or when the total scanned area amounts to a pre-selected portion of the target area.

Each of the foregoing optical imaging systems and methods of the present invention may incorporate analytical and/or numerical solution schemes disclosed in the commonly assigned co-pending U.S. non-provisional patent application bearing Ser. No. 09/664,972, entitled “A system and Method for Absolute Oxygen Saturation” by Xuefeng Cheng, Xiaorong X. u., Shuoming Zhou, and Ming Wang which has been filed on Sep. 18, 2000 and which is incorporated herein by reference in its entirety (referred to as “the '972 application” hereinafter). Such optical imaging system can calculate absolute values of the concentration of oxygenated hemoglobin, [HbO], the concentration of deoxygenated hemoglobin, [Hb], the oxygen saturation, SO₂, and the temporal changes in blood or water volume by adopting any of the solution schemes disclosed in the foregoing co-pending '972 application. Accordingly, such optical imaging system can provide the foregoing images that allow physicians to make direct diagnosis of the target area of the medium based on the “absolute” or “relative” values of the chromophores or their properties in the physiological media. In addition, operational characteristics of the optical imaging systems of the present invention incorporating any of the solution schemes disclosed in the above co-pending '972 application may not be affected by the number of the wave sources and/or detectors and by geometric configuration therebetween. Accordingly, the optical imaging systems of the present invention may include any number of wave sources and/or wave detectors arranged in any geometric arrangements.

As used herein, a “hemoglobin” or “hemoglobins” mean either or both of oxygenated hemoglobin and deoxygenated hemoglobin. The “hemoglobin,” “hemoglobins” or “values of hemoglobins” represent properties of such “hemoglobins.” Examples of such properties may include, but not limited to, amount or concentration thereof, total amount or concentration thereof (which corresponds to the sum of each amount or concentration of the oxygenated and deoxygenated hemoglobins), etc.

A “chromophore” refers to any substances in a physiological medium which can optically interact with electromagnetic waves transmitting therethrough. Chromophore generally includes solvents of a physiological medium, solutes dissolved in such a medium, and/or other substances included in the medium. Specific examples of such chromophores may include, but not limited to, cytochromes, enzymes, hormones, neurotransmitters, chemo- or chemical transmitters, proteins, cholesterols, apoproteins, lipids, carbohydrates, cytosols, cytosomes, blood cells, water, hemoglobins, and other optical materials present in animal or human cells, tissues or body fluid. Chromophores also include extra-cellular substances which may be injected into the medium for therapeutic and/or imaging purposes and may interact with electromagnetic waves. Such chromophores may include, but not limited to, dyes, contrast agents, and other image-enhancing agents, each of which may exhibit optical interaction with electromagnetic waves having wavelengths in a specific range.

“Electromagnetic waves” as used herein generally refer to acoustic or sound waves, near-infrared rays, infrared rays, visible light rays, ultraviolet rays, lasers, and/or rays of photons.

“Property” of the chromophores may mean intensive or extensive property thereof. Examples of such intensive property include, but not limited to concentration of the chromophore, a sum of such concentrations, and a ratio thereof. Examples of extensive property may include, but not limited to, volume, mass, weight, volumetric flow rate, and mass flow rate of the chromophores.

The term “value” is an absolute value of the chromophore property. The term “value” may also refer to a relative value representing spatial or temporal changes in the property of the chromophores including deoxygenated and oxygenated hemoglobins.

“Distribution” means two-dimensional or three-dimensional distribution of the values of the chromophores or their properties. The “distribution” may be measured or estimated in a spatial and/or temporal domain.

Unless otherwise defined, all technical and scientific terms used herein have the same meaning as commonly understood and/or used by one of ordinary skill in the art to which this invention belongs. Although methods and materials similar or equivalent to those described herein can be applied and/or used in the practice of or testing the present invention, suitable methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram of an optical imaging system according to the present invention;

FIG. 2 is a cross-sectional top view of an exemplary scanning unit according to the present invention;

FIG. 3 is a cross-sectional top view of another exemplary scanning unit according to the present invention;

FIG. 4 is a schematic diagram of an exemplary scanning unit arranged for linear translations according to the present invention;

FIG. 5 is a schematic diagram of another exemplary scanning unit arranged for rotation or revolution according to the present invention;

FIG. 6 is a schematic diagram of another exemplary scanning unit arranged for simultaneous X-translation and Y-reciprocation according to the present invention;

FIG. 7 is a schematic diagram of another exemplary scanning unit arranged to generate cross-voxels or cross measurement elements according to the present invention;

FIG. 8 is a schematic diagram of a mobile optical imaging system according to the present invention;

FIG. 9 is a schematic diagram of an optical imaging system according to the present invention;

FIGS. 10A and 10B are images of blood volume of normal and abnormal breast tissues, respectively, both of which are measured by the optical imaging system of FIG. 9 according to the present invention; and

FIGS. 11A and 11B are images of oxygen saturation of normal and abnormal breast tissues, respectively, both of which are measured by the optical imaging system of FIG. 9 according to the present invention.

### DETAILED DESCRIPTION OF THE INVENTION

The following description provides optical imaging systems for providing images of two- or three-dimensional spatial or temporal distributions of chromophores or their properties in a physiological medium. More particularly, the following description provides various aspects and preferred embodiments of such optical imaging systems and optical probes thereof equipped with movable scanning units or mobile source-detector assemblies.

In one aspect of the present invention, an optical imaging system provides images of distribution of chromophores or their properties in target areas of a physiological medium using a scanning unit which has a scanning area smaller than the target area.

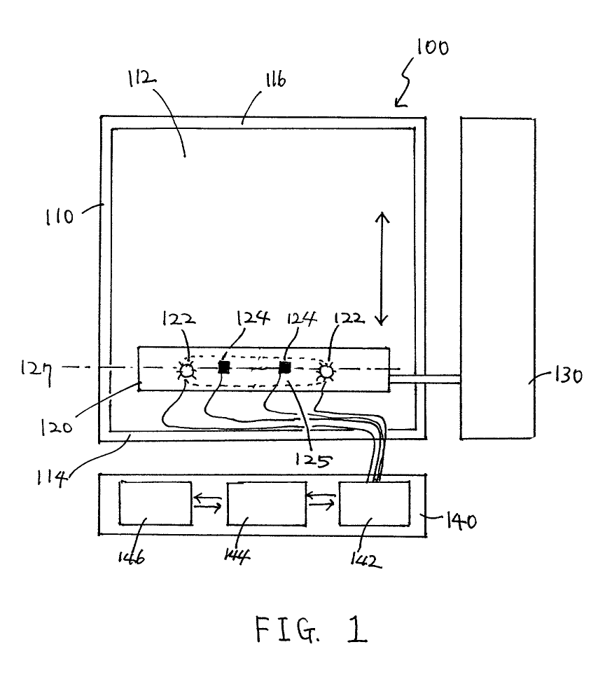

FIG. 1 is a schematic diagram of an optical imaging system according to the present invention. An exemplary optical imaging system 100 typically includes a body 110, movable member (or sensor assembly) 120, actuator member 130, and imaging member 140. Movable member 120 includes optical sensors such as multiple wave sources 122 and detectors 124. Actuator member 130 couples with movable member 120 and is arranged to move movable member 120 with respect to body 110 along one or more curvilinear paths in directions as shown by the arrows in the figure. Imaging member 140 operationally couples with the sensors (e.g., wave sources 122 and/or detectors 124) and generates images of the spatial or temporal distribution of the chromophores or their properties.

Body 110 includes a housing 112 substantially shaped as a rectangle and is arranged to receive movable member 120 therein. In general, housing 112 is shaped and sized substantially larger than movable member 120 so that movable member 120 can move along different portions of housing 112. As will be discussed below, the area of housing 112 generally corresponds to a “target area” of the medium that is to be scanned by sensors 122, 124 disposed at movable body (or sensor assembly) 120. Configuration of body 110 is generally determined according to various design criteria, e.g., the shape and size of the area of the medium to be scanned, shape and size of movable member 120, configuration of the curvilinear paths along which actuator member 130 moves movable member 120 across different regions or portions of the target area, etc. Body 100 may be made of semi-rigid or flexible material to conform to contoured surface of the medium.

Movable member 120 is generally elongated and includes a longitudinal axis 127. Movable member 120 also includes optical sensors such as wave sources 122 and detectors 124 each of which is aligned along longitudinal axis 127. Wave sources 122 are generally disposed at each end of movable member 120 and wave detector 124 interposed therebetween at equal distances so that electromagnetic waves emitted by wave sources 122 travel through the medium, interact with the medium, and are detected by wave detectors 124. Therefore, wave sources 122 and detectors 124 functionally form a scanning unit 125 (i.e., source-detector arrangement) that is elongated around wave sources 122 and detectors 124 along longitudinal axis 127 of movable member 120 and that defines a corresponding scanning area (or scanning volume). Movable member 120 may also be made of semi-rigid or flexible material so that sensors 122, 124 may form optical coupling while conforming to the surface contour of the target area.

The wave sources of the present invention are generally constructed to form optical coupling with the medium and to irradiate the electromagnetic waves thereinto. The movable member or sensor assembly may employ any wave sources that can irradiate the electromagnetic waves having pre-selected wavelengths, e.g., in the ranges from 100 nm to 5,000 nm, from 300 nm to 3,000 nm or, in particular, in the “near-infrared” range from 500 nm to 2,500 nm. As will be described below, however, typical wave sources are arranged to irradiate near-infrared electromagnetic waves having wavelengths about 670 nm to 710 nm, e.g., 690 nm, and about 810 nm to 850 nm, e.g., 830 nm. The wave sources may irradiate electromagnetic waves having different wave characteristics such as different wavelengths, phase angles, frequencies, amplitudes, harmonics, etc. In the alternative, the wave sources may irradiate electromagnetic waves in which identical, similar or different signal waves are superposed on carrier waves with similar or distinguishable wavelengths, frequencies, phase angles, amplitudes, and/or harmonics. In the embodiment of FIG. 1, each wave source 122 is arranged to irradiate near-infrared electromagnetic waves of two different wave lengths, e.g., about 690 nm and about 830 nm.

Similarly, the foregoing wave detectors are preferably arranged to detect the aforementioned electromagnetic waves and to generate the output signal in response thereto. Any wave detectors may be used in the movable member or sensor assembly as long as they have appropriate detection sensitivity to the electromagnetic waves having wavelengths in the foregoing ranges. The wave detector may also be constructed to detect electromagnetic waves which may have any of the foregoing wave characteristics. The wave detector may also detect multiple sets of electromagnetic waves irradiated by multiple wave sources and generate multiple output signals accordingly.

As discussed above, configuration of the scanning unit and scanning area thereof is predominantly determined by that of the movable member, sensor assembly or source-detector arrangement, e.g., the number of wave sources and detectors, geometric arrangement therebetween, irradiation capacity or emission power of the wave source, detection sensitivity of the wave detector, etc. In the embodiment shown in FIG. 1, wave sources 122 and detectors 124 define scanning unit 125 which is substantially elongated along longitudinal axis 127 of movable member 120. Although scanning unit 125 and/or movable member 120 may be characterized by any of its dimensions (e.g., length, width, and height), a “characteristic dimension” of scanning unit 125 is generally the one which is orthogonal to a direction in which scanning unit 125 and movable member 120 are moved by actuator member 130. Accordingly, as will be explained in greater detail below, the characteristic dimension of scanning unit 125 of FIG. 1 is its height. As shown in FIG. 1, scanning unit 125 preferably moves with movable member 120. Thus, unless otherwise specified, the terms “scanning unit” and “movable member” are to be used interchangeably throughout the disclosure.

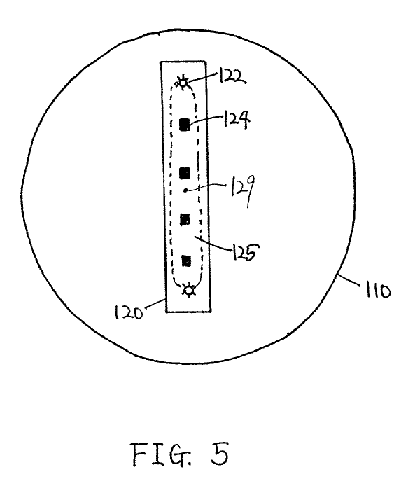

Actuator member 130 operationally couples with movable member 120 and linearly translates movable member 120 (along with wave sources 122 and detectors 124 from one side of body 110 to another side thereof in a direction normal to longitudinal axis 127 of movable member 120. In this embodiment, the width of movable member 120 is substantially similar to that of housing 112. Therefore, scanning unit 125 can scan through at least a substantial portion of the target area while being linearly translated across the target area. Any conventional actuating devices can be used as the actuator member of the optical imaging system of the present invention for generating movement of the movable member. For example, a stepper motor assembly is used to generate movements such as curvilinear translations, reciprocations, and a combination thereof. Specific examples of such curvilinear translations may include linear displacements along linear paths and non-linear translations along curved paths. Optional guiding tracks may be provided in and/or around the housing or the body to guide the movable member therealong. In the alternative, a motor-gear assembly may be used to generate rotational motion about a center of rotation around a pre-selected angle or for a pre-selected number of revolutions (see FIG. 5). The actuator member may also be arranged to impart various temporal characteristics to various movements of the movable member, e.g., by generating impulsive [i.e., functions of δ(t)], stepwise [i.e., functions of u(t)], pulse-train, and/or sinusoidal movements. In addition, the actuator member may be arranged to generate such movements continuously, periodically, and/or intermittently.

Imaging member 140 operationally couples with wave sources 122 and/or detectors 124 and is arranged to generate two- or three-dimensional images representing spatial or temporal distribution of the chromophores or their properties in the medium. As described in the figure, imaging member 140 typically includes a data acquisition unit 142 (i.e., signal acquisition unit or signal processor), algorithm unit 144, and image construction or image generation unit 146 (i.e., image processor). Data acquisition unit 142 is arranged to sample optical or electrical data or signals which are generated by sensors 122, 124 and are related to intensity, magnitudes, amplitudes or other characteristics of electromagnetic waves irradiated by wave source 122 and detected by wave detectors 124. Data acquisition unit 142 may also monitor other system variables or parameters related with the actuator member as well as an optional control member for controlling operation of each component of optical imaging system 100. Algorithm unit 144 receives various signals or data from data acquisition unit 142 and obtains solutions of the multiple wave equations applied to wave sources 122 and/or detectors 124. Conventional analytical or numerical schemes may be used in algorithm unit 144 to solve a set of wave equations such as the photon diffusion equation, Beer-Lambert equation, modified Beer-Lambert equation, and their equivalents. Algorithm unit 144 may then determine the absolute or relative values of the chromophores or their properties directly from such solutions or by further mathematical manipulations or signal processing thereof. Image construction unit 146 processes the foregoing absolute or relative values of the chromophores or their properties, and provides images for the two- or three-dimensional distribution pattern of the chromophores or their properties in the spatial and/or temporal domain.

In operation, movable member 120 is positioned in its starting position, e.g., a bottom portion 114 of housing 112. Body 110 of optical imaging system 100 is placed on the medium so that scanning unit 125 of movable member 120 is positioned in a first region of the target area. Wave sources 122 and detectors 124 are activated so that electromagnetic waves are emitted into and detected from the target area. Actuator member 130 is activated to translate movable member 120 substantially linearly from bottom 114 of housing 112 to a top 116 thereof in an upward direction which is normal to longitudinal axis 127 of movable member 120. During the upward linear translation, scanning unit 125 scans each region of the target area and wave detectors 124 generate output signals which are representative of the optical properties of the chromophores or their properties in each region of the target area. Once movable member 120 reaches top portion 116 of body 110, actuator member 130 moves movable member 120 back to its starting position (i.e., bottom portion 114) along the same path but in an opposite direction. During the downward linear translation, scanning unit 125 again sweeps through the similar or different regions of the same target area and wave detectors 124 generate the output signals. The foregoing scanning procedure may be completed after movable member 120 finishes the foregoing reciprocation across the target area.

The optical imaging system of the present invention offers numerous benefits over the prior art optical imaging technology such as the near-infrared spectroscopy, diffuse optical spectroscopy, and other conventional optical sensors or imaging equipment. Prior art optical sensor assemblies generally define scanning units designed to make only a single measurement in each measurement location. Accordingly, when the area of a subject to be examined is larger than the scanning area of the scanning unit, the prior art scanning units must be manually moved to different locations of the subject, and multiple measurements must be made at different measurement locations thereof. Such prior art procedure tends to lengthen examination periods, not to mention the final images with poor resolution due to inaccurate positioning of the sensors in different measurement locations of the subject or inconsistent optical coupling between the medium and sensors at different measurement locations. In order to rectify such deficiencies, bigger sensor assemblies with a far greater number of wave sources and detectors have been developed so that they can cover a larger target area in each measurement. However, such sensor assemblies are generally bulky and more expensive. In addition, idiosyncratic variation among the sensors jeopardizes quality of the optical and/or electrical output signals, thereby degrading quality of resulting images.

The optical imaging system of the present invention overcomes the foregoing prior art deficiencies by providing a scanning unit which includes only a minimal number of wave sources and detectors but which is movably constructed so that it can sweep through a much larger target area without having to move and reposition the movable member and/or optical probe of the optical imaging system. Such optical imaging system allows scanning of the larger target area by the scanning unit having the scanning area which is only a small fraction of the target area. The foregoing optical imaging system requires fewer number of sensors (i.e., only a minimal number of wave sources and/or detectors) than their prior art counterparts, thereby allowing construction of compact and light optical probes or optical imaging systems. In addition, idiosyncratic discrepancies attributed to component variances inherent in each sensor may be minimized, thereby providing final images with improved accuracy, high-quality, and high-resolution. The foregoing optical imaging system further facilitates maintaining consistent optical coupling between the medium and sensors during the movement of the movable member. Accordingly, the foregoing optical imaging system may establish a single baseline of the output signal and apply such a baseline throughout different regions of the target area and/or different target areas of the entire medium. The optical imaging system of the present invention also allows use of a more efficient image construction scheme for providing real-time images of the distribution of the chromophores or their properties while scanning the target area of the test subject.

Although any analytical and/or numerical schemes may be employed, the algorithm unit of the present invention preferably incorporates solution schemes disclosed in the co-pending '972 application. For example, the absolute values of concentration of deoxygenated hemoglobin, [Hb], concentration of oxygenated hemoglobin, [HbO], and oxygen saturation, SO₂, are obtained by the following equations (1a) to (1e) each of which corresponds to the equations (8a) through (8d) and (9b) of the co-pending '972 application, respectively:

$$\begin{matrix}[ Hb ] = \frac{{ϵ}_{HbO}^{{λ}_{2}} \frac{{OD}^{{λ}_{1}}}{{F}^{{λ}_{1}}} - {ϵ}_{HbO}^{{λ}_{1}} \frac{{OD}^{{λ}_{2}}}{{F}^{{λ}_{2}}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(1a)} \\ [ HbO ] = \frac{{ϵ}_{Hb}^{{λ}_{1}} \frac{{OD}^{{λ}_{2}}}{{F}^{{λ}_{2}}} - {ϵ}_{Hb}^{{λ}_{2}} \frac{{OD}^{{λ}_{1}}}{{F}^{{λ}_{1}}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(1b)}\end{matrix}$$

F^{λ1}=(B_{S1D2}^{λ1}L_{S1D2}=B_{S1D1}^{λ1}L_{S1D1})+(B_{S2D1}^{λ1}L_{S2D1}=B_{S2D2}^{λ1}L_{S2D2}) (1c)

F^{λ2}=(B_{S1D2}^{λ2}L_{S1D2}=B_{S1D1}^{λ2}L_{S1D1})+(B_{S2D1}^{λ2}L_{S2D1}=B_{S2D2}^{λ2}L_{S2D2}) (1d)

$${SO}_{2} = \frac{{ϵ}_{Hb}^{{λ}_{1}} \frac{{OD}^{{λ}_{2}}}{{OD}^{{λ}_{1}}} \frac{{F}^{{λ}_{1}}}{{F}^{{λ}_{2}}} - {ϵ}_{Hb}^{{λ}_{2}}}{( {ϵ}_{Hb}^{{λ}_{1}} - {ϵ}_{HbO}^{{λ}_{1}} ) \frac{{OD}^{{λ}_{2}}}{{OD}^{{λ}_{1}}} \frac{{F}^{{λ}_{1}}}{{F}^{{λ}_{2}}} + ( {ϵ}_{HbO}^{{λ}_{2}} - {ϵ}_{Hb}^{{λ}_{2}} )} \qquad \text{(1e)}$$

where the parameters “ε_{Hb}” and “ε_{HbO}” represent extinction coefficients of the deoxygenated and oxygenated hemoglobins, respectively, the variable “OD” is an optical density defined as a logarithmic ratio of light intensities (i.e., magnitudes or amplitudes of electromagnetic waves) detected by a wave detector, the parameter “B” is conventionally known as a “path length factor,” the parameter “L_{S1Dj}” is a distance between an i-th wave source and a j-th wave detector, and the superscripts “λ₁” and “λ₂” represent that a parameter or variable is obtained by irradiating electromagnetic waves having wavelengths λ₁ and λ₂, respectively.

In the alternative, the algorithm unit of the imaging member may employ the over-determined iterative method as disclosed in the foregoing '972 application, where the absolute values of [Hb], [HbO], and SO₂ are determined by the following equations (2a) to (2c), each of which corresponds to the equations (17a) through (17c) of the co-pending '972 application, respectively:

$$\begin{matrix}[ Hb ] = \frac{{ϵ}_{HbO}^{{λ}_{2}} {μ}_{a}^{{λ}_{1}} - {ϵ}_{HbO}^{{λ}_{1}} {μ}_{a}^{{λ}_{2}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(2a)} \\ [ HbO ] = \frac{{ϵ}_{Hb}^{{λ}_{1}} {μ}_{a}^{{λ}_{2}} - {ϵ}_{Hb}^{{λ}_{2}} {μ}_{a}^{{λ}_{1}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(2b)} \\ {SO}_{2} = \frac{[ HbO ]}{[ Hb ] + [ HbO ]} = \frac{{ϵ}_{Hb}^{{λ}_{1}} {μ}_{a}^{{λ}_{2}} - {ϵ}_{Hb}^{{λ}_{2}} {μ}_{a}^{{λ}_{1}}}{( {ϵ}_{HbO}^{{λ}_{2}} {μ}_{a}^{{λ}_{1}} - {ϵ}_{HbO}^{{λ}_{1}} {μ}_{a}^{{λ}_{2}} ) + ( {ϵ}_{Hb}^{{λ}_{2}} {μ}_{a}^{{λ}_{2}} - {ϵ}_{Hb}^{{λ}_{2}} {μ}_{a}^{{λ}_{1}} )} & \text{(2c)}\end{matrix}$$

where the parameter “μₐ” denotes an absorption coefficient of the medium. It is noted that the imaging member of the present invention may be arranged to receive the output signals generated by the wave detectors and to calculate optical densities which may be supplied to the algorithm unit. Once the absolute values of and/or their changes in the hemoglobins are determined, the imaging member generates images representing two- or three-dimensional spatial and/or temporal distributions of the hemoglobins by employing a real-time image construction technique which is discussed in greater detail in another commonly assigned co-pending non-provisional U.S. Patent Application bearing Serial No. (N/A), entitled “Optical Imaging System for Direct Image Construction” which has been filed on Feb. 5, 2001 and which is incorporated herein in its entirety by reference.

In the alternative, changes in the hemoglobin distribution are determined by estimating changes in optical characteristics of the target area of the medium. For example, changes in concentrations of oxygenated and deoxygenated hemoglobins may be calculated from the differences in their extinction coefficients which are measured by electromagnetic waves having two different wavelengths. In an exemplary numerical scheme, the photon diffusion equations may be modified and solved by the diffusion approximation described in, e.g., Keijer et al., “Optical Diffusion in Layered Media,” Applied Optics, vol. 27, p.1820-1824 (1988) and Haskell et al., “boundary Conditions for Diffusion Equation in Radiative Transfer,” Journal of Optical Society of America, A, vol. 11, p.2727-2741, 1994:

$${[ \begin{matrix}{Φ}_{SC} ( {r}_{Si} , {r}_{Di} ) \\ ⋮ \\ {Φ}_{SC} ( {r}_{SM} , {r}_{DM} )\end{matrix} ]}_{M , i} = {[ \begin{matrix}{W}_{11} & ⋯ & {W}_{i , N} \\ ⋮ & ⋰ & ⋮ \\ {W}_{Mi} & ⋯ & {W}_{MN}\end{matrix} ]}_{M , N} \cdot {[ \begin{matrix}{Δμ}_{a , i} \\ ⋮ \\ {Δμ}_{a , N}\end{matrix} ]}_{N , i} \qquad ( 3 )$$

where the symbol “Φ_{SC}(r_{Si}, r_{Dj})” represents a normalized optical density measured by a j-th wave detector in response to an i-th wave source, the variables “r_{Si}” and “r_{Dj}” are positions of the i-th wave source and j-th wave detector, respectively, the symbol “Δμₐ₄” denotes tissue optical perturbation such as the changes in the absorption coefficient in an i-th voxel, the parameters “M” and “N” refer to the number of measurements and the voxel number to be reconstructed, respectively, and the variable “Wᵢⱼ” is a weight function which represents the probability that a photon travels from the i-th wave source to a certain point inside the target area of the medium and is then detected by the j-th wave detector. The weight function, Wᵢⱼ, of equation (3) is defined as:

$${W}_{ij} = \frac{G ( {r}_{Di} , {r}_{j} ) \cdot {Φ}_{0} ( {r}_{Si} , {r}_{j} ) \cdot v \cdot {h}^{3}}{{D}_{photon}} \qquad ( 4 )$$

where the parameters “h³” is the volume of a voxel, “Dₚₕₒₜₒₙ” represents a photon diffusion coefficient, and “v” denotes the velocity of light in the physiological medium. In addition, the variable “Φ_{SC}(r_{Si}, r_{Dj})” is the normalized optical density defined as:

$${Φ}_{SC} ( {r}_{sI} , {r}_{Dj} ) = \frac{{I}_{B} - I}{{I}_{B}} \qquad ( 5 )$$

where the variable “I” represents the output signal measured by the sensor assembly which is comprised of the i-th wave source and j-th wave detector disposed at positions “r_{Si}” and “r_{Dj},” respectively, and the variable “I_{B}” denotes a baseline of the output signal detected by the wave detector.

Various methods such as, e.g., the direct matrix inversion and simultaneous iterative reconstruction techniques, may be applied to solve the above set of equations (3) to (5). Once the tissue optical perturbations, “Δμₐ^{λ1}” and “Δμₐ^{λ2}” are estimated by irradiating electromagnetic waves having two different wavelengths, λ₁ and λ₂, respectively, changes in concentrations of oxygenated hemoglobin and deoxygenated hemoglobin can be obtained as follows:

$$\begin{matrix}Δ [ Hb ] = \frac{{ϵ}_{HbO}^{{λ}_{2}} \cdot {Δμ}_{a}^{{λ}_{1}} - {ϵ}_{HbO}^{{λ}_{1}} \cdot {Δμ}_{a}^{{λ}_{2}}}{( {ϵ}_{Hb}^{{λ}_{1}} {ϵ}_{HbO}^{{λ}_{2}} - {ϵ}_{HbO}^{{λ}_{1}} {ϵ}_{Hb}^{{λ}_{2}} ) \cdot L} & \text{(6a)} \\ Δ [ HbO ] = \frac{{ϵ}_{Hb}^{{λ}_{1}} \cdot {Δμ}_{a}^{{λ}_{2}} - {ϵ}_{Hb}^{{λ}_{2}} \cdot {Δμ}_{a}^{{λ}_{1}}}{( {ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}} ) \cdot L} & \text{(6b)}\end{matrix}$$

where L is the distance between the wave source and detector and where the parameters ε_{Hb}^{λ1}, ε_{Hb}^{λ2}, ε_{HbO}^{λ1}, and ε_{HbO}^{λ2} represent extinction coefficients of oxygenated and deoxygenated hemoglobins measured at two different wavelengths, λ₁ and λ₂, respectively.

Incorporating any of the foregoing solution schemes into the optical imaging system of the present invention offers additional benefits over the prior art optical imaging technology. Contrary to the CWS allowing measurement of changes in the hemoglobin concentrations, the foregoing optical imaging systems provide a direct means for assessing spatial or temporal distribution of the “absolute values” of hemoglobins in the physiological medium. This allows a physician to measure oxygen concentrations and oxygen saturation in tissues, cells, organs, muscles, blood or body fluids of an animal or human subject. The optical imaging system of the present invention also allows the physician to make a direct diagnosis based on the “absolute values” of the hemoglobin concentrations and the oxygen saturation of the medium.

As discussed above, the embodiment disclosed in FIG. 1 is only an example of the optical imaging system of the present invention including the movable scanning unit. The foregoing optical imaging system may be modified without departing from the scope of the present invention.

It is noted that the exact number of the wave sources and detectors included in the movable member is not critical in realizing the objective of the present invention. For example, the movable member may include only a single wave source capable of irradiating multiple sets of electromagnetic waves having, e.g., different wave characteristics, identical or different signal waves superposed on different or identical carrier waves, etc. In addition, such wave sources may irradiate such electromagnetic waves continuously, periodically or intermittently. Similarly, the movable member may include only a single wave detector that can detect the foregoing electromagnetic waves continuously, periodically or intermittently.

The scanning unit preferably defines a continuous scanning area so that the scanning unit generates the output signal covering the entire region of the scanning area without any interruption due to unscanned regions. For this purpose, the wave sources and detectors may be spaced at distances no greater than a threshold distance determined by the irradiation capacity of the wave source, detection sensitivity of the wave detector, and the like. Selection of the optimal spacing between the wave source and detector is generally a matter of choice of one of ordinary skill in the art and such spacing may be determined by several factors including, but not limited to, optical properties of the physiological medium (i.e., absorption coefficient, scattering coefficient, and the like), number of wave sources and/or detectors, geometric arrangement therebetween, grouping of the wave sources and detectors in each of the scanning units, operational characteristics of the actuator member, and the like.

The actuator member may generate at least two movements of the movable member sequentially or simultaneously along at least two curvilinear paths in at least two curvilinear directions. The actuator member may move the movable member along the curvilinear paths substantially orthogonal to each other, e.g., as signified by the orthogonal axes of the Cartesian, cylindrical, and/or spherical coordinate systems. Alternatively, such movements may take place along the identical or parallel curvilinear paths in exact opposite directions as exemplified in the reciprocating movements.

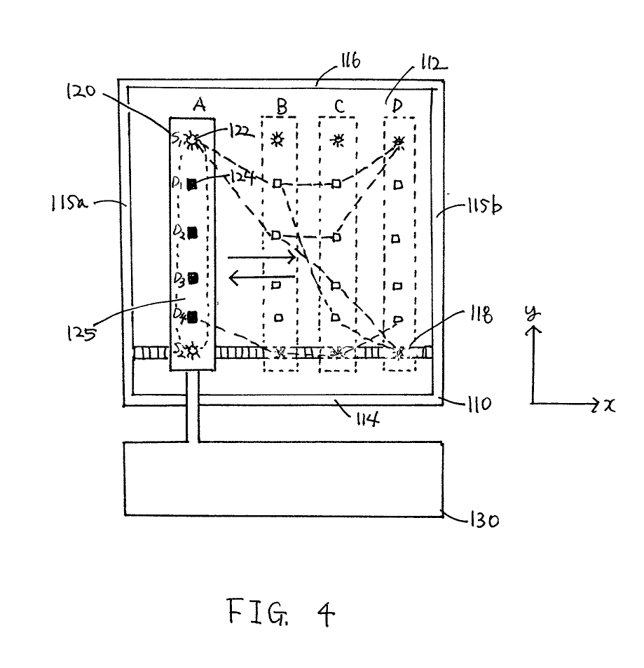

The movable body, scanning unit thereof, and/or actuator member may be arranged to attain various geometric configurations with respect to the longitudinal axis of the movable member as well as to the curvilinear paths generated by the actuator member. As shown in FIG. 1, the actuator member may be aligned to move the movable member in a direction which is substantially transverse to the longitudinal axis of the movable member. Conversely, the actuator member may move the movable member along a path substantially parallel or at an angle with respect to the longitudinal axis of the movable member. It is preferred, however, that the actuator member should move the movable member so that the curvilinear path of the movable member is orthogonal to a longitudinal axis of the scanning unit (i.e., the axis connecting the wave sources and detectors). This embodiment maximizes the effective scanning area of the scanning unit during movements of the movable member. In another embodiment, the actuator member may generate the foregoing movements at a constant speed or at speeds which may vary over time or position over the target area. An optional motion controller may be provided so that the speed of such movement may be controlled precisely according to a pre-determined pattern. Alternatively, such movement may be controlled adaptive to the operational parameters such as, e.g., optical characteristics of the medium and presence or absence of abnormal lesions in the target area which may be signified by, e.g., abnormally high or low absorption or scattering of electromagnetic waves transmitted therethrough. Further details of the actuator member will be provided below in conjunction with the exemplary embodiments of the scanning units as illustrated in FIGS. 4 through 8.

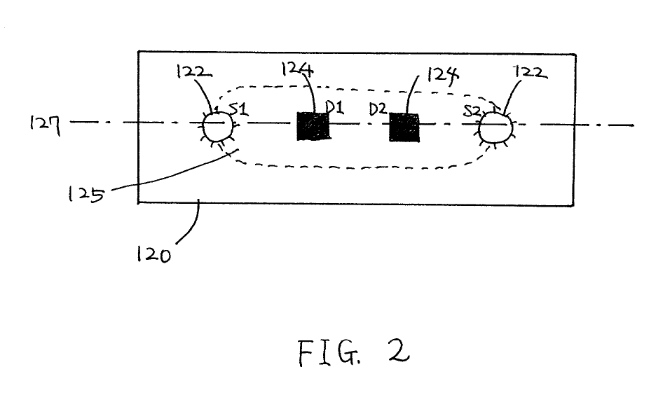

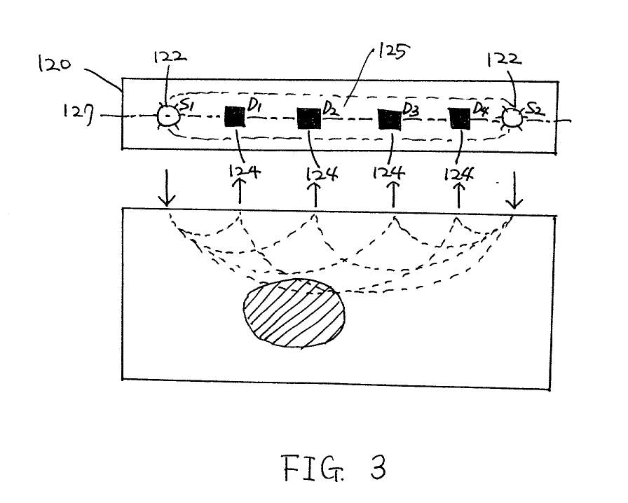

The optical imaging system of the present invention determines absolute or relative values of the chromophores or their properties by obtaining the set of solutions of multiple wave equations using one of the solution schemes disclosed in the co-pending '972 application. Therefore, as long as the wave sources and detectors satisfy the requirements of identical near-distances and far-distances (i.e., “symmetry requirements”) disclosed in the co-pending '972 application, operational characteristics of the foregoing optical imaging systems of the invention become generally insensitive to the number of wave sources and detectors included in the movable member as well as to configuration of the scanning unit. Accordingly, the optical imaging system of the present invention can include any number of wave sources and/or detectors arranged in any geometric configurations, subject only to the “symmetric requirements” of the co-pending '972 application. However, the scanning unit may preferably be constructed according to a few semi-empirical design rules which are expected to provide enhanced accuracy, reliability, and/or reproducibility of the estimated chromophore properties and/or images thereof. These exemplary design rules are: (1) the scanning unit preferably includes at least two wave sources and at least two wave detectors; and (2) the distance between adjacent wave source and detector preferably does not exceed a threshold sensitivity range of the wave detector which may range from, e.g., several cm to 10 cm or, in particular, about 5 cm for most human and animal tissues or cells. FIGS. 2 and 3 describe a few exemplary embodiments of the scanning units constructed according to the foregoing design rules.

FIG. 2 is a cross-sectional top view of an exemplary movable member and scanning unit thereof according to the present invention. Contrary to conventional source-detector arrangements where each wave source is surrounded by multiple wave detectors or vice versa, scanning unit 125 of FIG. 2 is defined by two wave sources 122 (i.e., S₁ and S₂) which are disposed along longitudinal axis 127 of movable member 120 as well as by two wave detectors 124 (i.e., D₁ and D₂) which are interposed between wave sources 122 along the same axis 127. In particular, adjacent wave source 122 and detector 124 are spaced at substantially equal distances. Therefore, scanning unit 125 and the scanning area defined thereby are elongated along the same axis 127 and have a characteristic “height” which is determined by, e.g., irradiation capacity or emission power of wave sources 122, detection sensitivity or range of wave detectors 124, optical characteristics of the medium, and the like.

It is appreciated that scanning unit 125 of FIG. 2 satisfies the “symmetry requirements” disclosed in the co-pending '972 application, i.e., the wave sources and detectors are arranged to maintain similar or substantially identical near- and far-distances therebetween during the movement of the movable member. For example, the first near-distance between the wave source S₁ and detector D₁ is identical or substantially similar to the second near-distance between the wave source S₂ and detector D₂. In addition, the first far-distance between the wave source S₁ and detector D₂ is identical or substantially similar to the second far-distance between the wave source S₂ and detector D₁. A major advantage by meeting such symmetric arrangements lies in the observation that electromagnetic waves are substantially uniformly transmitted, absorbed or scattered throughout the entire target area or target volume of the medium. Accordingly, scanning unit 125 uniformly covers all regions of the target area of the medium and, therefore, enhances the accuracy and reliability of the output signal (e.g., an improved signal-to-noise ratio) generated by the wave detector.

It is further appreciated that scanning unit 125 of FIG. 2 employs the source-detector arrangement that is completely contrary to the conventional norms for constructing the optical probes. For example, the conventional scanning units include a large number of wave sources and detectors so that the target area of the medium is substantially identical to the scanning area of such prior art probes. To the contrary, the optical imaging system of the present invention has significantly fewer wave sources and detectors which are aligned substantially along the longitudinal axis of the movable member. Such one-dimensional linear arrangement would be a fatal drawback for the conventional probes, for the scanning area defined by the linearly aligned sensors only amounts to a narrow strip of the target area. However, the optical imaging system of the present invention includes the actuator member arranged to generate various movements of the movable member and scanning unit thereof to cover every region of the target area. Accordingly, the foregoing linear scanning unit of the optical imaging system of the present invention can be significantly narrower than the target area of the medium. Further benefits and advantages of such embodiments will be discussed in conjunction with exemplary embodiments which are explained in greater detail below.

FIG. 3 is a cross-sectional top view of another exemplary movable member and its scanning unit according to the present invention. Scanning unit 125 includes two identical wave sources 122 disposed along longitudinal axis 127 of movable member 120 and four identical wave detectors 124 interposed between wave sources 122 and aligned along the same axis 127 at substantially equal distances. The embodiment of FIG. 3 is different from that of FIG. 2 in a few aspects. First, not every source-detector arrangement of scanning unit 125 of FIG. 3 satisfies the identical near- and far-distance configuration of FIG. 2. For example, although the first and fourth wave detectors (D₁ and D₄) as well as the second and third wave detectors (D₂ and D₃) have the identical near- and far-distances from wave sources 122, such distances are different for the first and third wave detectors (D₁ and D₃) or the second and fourth wave detectors (D₂ and D₄). In addition, banana-shaped paths (see the figure) of electromagnetic waves also reveal that each source-detector arrangement covers different portions of the target area and, therefore, detects the electromagnetic waves absorbed and/or scattered through different regions of the target area in different extents. However, by linearly interposing wave detectors 124 between wave sources 122, all regions of the target area are substantially uniformly covered in appropriate depth into the medium. Furthermore, scanning unit 125 of FIG. 3 can provide a longer scanning area because it includes more wave detectors 124 and, therefore, extends farther along longitudinal axis 127 of movable member 120 than the one in FIG. 2. Therefore, scanning unit 125 of FIG. 3 can cover the longer and possibly wider region of the target area per each measurement.

The foregoing movable member and scanning unit thereof may be modified without departing from the scope of the present invention. For example, three or more wave sources (or detectors) may be incorporated into the movable member, where at least two or all of the wave sources (or detectors) are disposed substantially linearly along the longitudinal axis of the movable member. The wave detectors (or sources) may also be interposed between two or more wave sources (or detectors) along the longitudinal axis of the movable member. Alternatively, the movable member may include at least two wave sources (or detectors), where the first wave source (or detector) is disposed on one side across the longitudinal axis of the movable member, while the second wave source (or detector) is disposed on the other side across the same axis thereof. Such wave sources (or detectors) may also be disposed symmetrically with respect to the longitudinal axis of the movable member.

In another aspect of the invention, an optical imaging system includes at least one of the foregoing wave sources and at least one of the foregoing wave detectors each of which directly couples with a body of the system which in turn may be either stationary or mobile. Except the absence of the movable member therein, such optical imaging system may be made substantially similar to those of FIGS. 1 to 3, e.g., including body 110, sensor assembly (which corresponds to movable member 120 of FIG. 1) having at least one wave source 122 and at least one wave detector 124, actuator member 130 for generating at least one of the foregoing movements of sensor assembly 120 and/or body 110 relative to the target area, and imaging member 140 for receiving signals from sensor assembly 120 and generating the images of the distribution of the chromophores or their properties.

In one embodiment, the sensor assembly may include the foregoing wave sources and detectors and be fixedly coupled to a scanning surface of the body, while the body itself is arranged to be movable with respect to the target area and other parts of the optical imaging system. Because the wave sources and detectors are fixedly coupled with the sensor assembly, a single movement of the body results in the movement of the sensor assembly and the body in unison. Therefore, the sensors can maintain a constant geometric arrangement therebetween with respect to the body during the movement thereof. This embodiment offers the benefit of simple mechanical construction and enhanced support attained by the fixed coupling between the sensor assembly and the body.

In another embodiment, the sensor assembly may not fixedly couple with the body. Rather, the actuator member generates separate movements of the sensor assembly and the body so that each of the sensor assembly and the body moves with respect to the other as well as to the target area. Despite complicated design and control requirements, this embodiment provides the sensor assembly with greater flexibility in scanning different regions of the target area along a variety of curvilinear movement paths thereof.

Other embodiments disclosed in conjunction with FIGS. 1 through 3 may be applied to this aspect of the invention. For example, the actuator member may generate one or more movements continuously, intermittently or periodically. The actuator member may generate such movement at a constant speed or at speeds varying over time and/or position over the target area. Alternatively, the actuator member may further be arranged to generate two or more of the foregoing movements simultaneously or sequentially.

In yet another aspect of the invention, an optical imaging system includes at least one of the foregoing wave sources, at least one of the foregoing wave detectors, an actuator member, at least one optical probe including a movable member therein, a console (or main body), and a connector member. The actuator member generates the movement of the wave source, wave detector, and/or movable member along at least one curvilinear path. The connector member provides optical, electrical or mechanical communication between the probe and console. For example, the connector member may include power lines and/or electric wire to transmit electric power or electrical (analog and/or digital) signals or data. The connector member may also include optical pathways such as an fiber optics article (e.g., an optical fiber) to transmit electromagnetic waves between the probe and console. Furthermore, the connector member may provide mechanical support between the probe and console and/or transmit translating, rotating, revolving or reciprocating motion generated by the actuator member of the console to the movable member through the power transmission pathways such as a flexible power cable or universal joint.

In one embodiment, the movable member of the optical probe includes at least one wave source and at least one wave detector. Electric power is preferably supplied by an internal power mechanism of the optical probe and/or from the console through the connector member. The actuator member may be implemented to the optical probe to move the movable member. Alternatively, the actuator member is disposed in the console where translational, rotational, revolving, and/or reciprocating motion may be transmitted to the movable member through the connector member. Some or entire portion of the imaging member may similarly be disposed at either or both of the optical probe and console.

In another embodiment, the console includes the wave sources and detectors, whereas the movable member of the optical probe includes minimum instrumentation such that the movable member can receive the electromagnetic waves generated by the wave sources, transmit the waves into the target area of the medium, collect the waves emanating from the target area, and/or transmit the electromagnetic waves to the wave detectors of the console. In one exemplary embodiment, the movable member defines two apertures on its scanning surface, where a first optical fiber is disposed between the wave source and the first aperture and where the second optical fiber is disposed between the wave detector and the second aperture. By arranging the first and second apertures to form appropriate optical coupling with the medium, the target area can be indirectly scanned by the wave sources and detectors disposed at the console. Similar to the foregoing embodiment, electric power may be supplied to the optical probe by an internal power mechanism of the optical probe or from an external or main power mechanism of the console through the connector member. The actuator member is disposed at the optical probe to move at least one of the first and second apertures which optically couple with the wave source and detector of the console as discussed above. In the alternative, the actuator member may be disposed in the console and the translational, rotational, revolving or reciprocating power generated by the actuator member is transmitted to the movable member through the connector member. Similarly, the imaging member may be disposed at either or both of the optical probe and the console.

In either embodiment, an optional display may be provided to the optical probe so as to allow an operator to view raw images (e.g., distribution patterns of system variables such as the output signal generated by the wave detector), processed images (e.g., distribution patterns of functions or solutions obtained by processing the raw signal), and/or final images (e.g., distribution patterns of chromophores or their properties). The optical probe may also include a data transmission unit through which any of the foregoing signals or data may be transmitted to the imaging member telemetrically or through the connector member on a real time or on an intermittent or periodic basis.

The foregoing embodiment of this aspect of the invention is also beneficial over the prior art technologies. First, bulky or heavy components such as a power supply, wave generator (e.g., a lamp, laser source or drive, and the like), photo-detector, detector drive, and/or circuit boards, are incorporated into the console, while only essential elements (e.g., optical fibers) are disposed in the portable probe. Thus, the optical probe is provided as a compact and light weight article. Secondly, idiosyncratic errors due to the component variances are also minimized because such optical probe needs fewer components than its prior art counterparts. Thirdly, the foregoing optical probe may be constructed as a semi-portable article wearable by a test subject for continuous or periodic monitoring or imaging of the target area.

In a further aspect of the invention, an optical imaging system includes at least one portable probe and a console (or main body). Such a portable probe is provided with at least one movable member and an actuator member both of which are substantially similar to those described hereinabove so that the actuator member moves the movable member along at least one curvilinear path. The console generally includes an imaging member. In one embodiment, the portable probe and console are operationally connected by a connector member which may provide the foregoing communications between the portable probe and console. In another embodiment, the portable probe may be provided as a separate article which is physically detachable from the console. Such a portable probe preferably includes at least one wave source, at least one wave detector, an actuator member such as a miniature motor assembly, and an internal power mechanism capable of supplying electric power to the above components of the portable probe. In addition, the portable probe may include a signal or data storage unit and/or signal or data transmission unit so that the signal or data may be temporarily stored therein or telemetrically transmitted to the console. The portable probe may include a separate imaging member so as to generate two- or three-dimensional raw images, processed images or final images of the chromophore or their properties in the target area The internal power mechanism is preferably rechargeable and has the capability of sustaining the portable probe for a pre-determined period. The primary advantage of this embodiment lies in the fact that such portable probe may be worn by a test subject or even be implanted inside the subject for constant or periodic monitoring and/or imaging of the target area thereof.

In yet another aspect of the invention, an optical imaging system includes two or more wave sources and two or more wave detectors, where at least two of the wave sources and at least two of the wave detectors are substantially linearly disposed along a straight line. Such wave sources and detectors generally define an elongated scanning unit having an elongated scanning area which is substantially narrower than the target area. By incorporating an actuator member which generates any of the foregoing movements of at least one of the wave sources and detectors, such optical imaging system enables scanning of the entire target area by the movable scanning unit sized as only a small fraction of the target area.

The foregoing aspect of the present invention offers further benefits over conventional optical imaging technologies. Prior art optical imaging equipment typically relies on a single, large optical probe designed to cover the entire portion of the target area. Therefore, the prior art probe has to include a large number of wave sources and detectors disposed on its sensing area. With such a large number of wave sources and detectors, the prior art technology suffers from various disadvantages. First, such probe is generally big and bulky. Thus, unless the probe is arranged to conform to the curvature of the target area, some wave sources and/or detectors may be subject to form poor optical coupling with the contoured target area. Even if the probe is provided with a conforming surface, such target-specific probe may find limited utility. In addition, the output signals and final images generated by such probe include a significant amount of noise attributed to idiosyncratic variances among the sensors. To the contrary, the optical imaging system of the present invention typically defines the scanning unit comprised of fewer sensors many or all of which may be linearly aligned along the longitudinal axis of the scanning unit. Therefore, the scanning unit forms a narrow sensor strip which more easily conforms to the surface contour of the target area. In addition, by arranging the actuator member to translate or rotate the slim scanning unit in different regions of the target area, the foregoing optical imaging system scans the entire target area while maintaining consistent optical coupling with the target area. The foregoing optical imaging system also requires fewer sensors, thereby reducing manufacturing cost as well as minimizing the noises attributed to the idiosyncratic component variances.

As discussed herein, the actuator member generates the movements of the movable member and its scanning unit so that the smaller scanning unit can cover a larger target area. Following figures illustrate exemplary arrangements of the actuator member designed to generate the movements of the movable member. For illustration purposes only, the embodiment shown in FIG.3 has been selected as the exemplary scanning unit throughout FIGS. 4 through 6.

FIG. 4 is a schematic diagram of the scanning unit of FIG. 3 arranged for linear translations according to the present invention. Movable member 120 includes two identical wave sources 122 and four identical, equi-spaced wave detectors 124 interposed between wave sources 122. Therefore, scanning unit 125 is substantially elongated and extends along longitudinal axis 127 of movable member 120. Stationary body 110 is sized to be slightly larger than the desired target area of the medium so that body 110 can cover the entire target area. Body 110 has a rectangular (or square) shape and accommodates a length or height of elongated scanning unit 125. Body 110 also includes a linear guiding track 118 extending across an entire length of housing 112. Movable member 120 is movably disposed on guiding track 118 and guided thereby during the linear translation of movable member 120. It is noted that guiding track 118 is disposed inside housing 112 so that the presence of guiding track 118 does not interfere with movement of scanning unit 125 across different regions of the target area. Actuator member 130 such as a stepper motor assembly generates linear translational movement of scanning unit 125 along the linear path aligned substantially parallel with a top 116 and a bottom 114 of rectangular body 110. It is noted that at least a portion of body 110 may form a dead area or blind spot where scanning unit 125 cannot make any reliable measurements. In general, such dead area is confined to regions adjacent to edges and/or comers of body 110, and the size of the dead area generally depends on, e.g., a distance from an edge or comer of body 110 to wave sources 122 or detectors 124. Because the dead area generally wastes valuable real estate of body 110, it is preferably minimized by, e.g., conforming the shape of body 110 to the size and shape of scanning unit 125 as well as its curvilinear movement paths.

In operation, movable member 120 is positioned in a starting region which is generally adjacent to one side of body 110, e.g., left side 115a of housing 112. Body 110 is also positioned on the target area and wave sources 122 and detectors 124 are disposed on a surface of a first region of the target area to form optical coupling therewith. Wave sources 122 and detectors 124 are activated so that electromagnetic waves are irradiated into and detected from the target area. Actuator member 130 is activated and movable member 120 is linearly translated away from the first region to an adjacent second region of the target area. During the linear translation, wave sources 122 and detectors 124 are manipulated to maintain optical coupling with the medium so that wave detectors 124 can continuously generate output signals representing spatial or temporal distribution of the chromophores or their properties in the first, second, and other adjacent regions of the target area. The output signals and/or other signals for system variables or parameters (e.g., optical density signals, solution signals, distribution signals, image signals, and the like) are relayed to the imaging member which determines distribution of absolute or relative values of the chromophores or their properties in pixels (referred to as “voxels” hereinafter) in the image domain. Once movable member 120 reaches the opposing side of the target area, movable member 120 is translated back from the far-right region of the target area toward the starting, first region thereof. The imaging member determines another distribution of the absolute or relative values of the chromophore properties for the same or different set of voxels during this second movement. Depending on required resolution of the final images, this translation may be repeated for a pre-determined period or for a pre-selected number of repetitions. Wave sources 122 and detectors 124 are then moved to the starting position in the same region of the target area or to an adjacent different region of the target area, and scan the region by irradiating electromagnetic waves thereinto, by detecting electromagnetic waves therefrom, and by generating another set of output signals. After completing the scanning process, the imaging member removes the high frequency noise from the output signals, reorganizes the voxels to provide two-dimensional or three-dimensional spatial or temporal distribution of the chromophore properties, and generates the images of the spatial and/or temporal distribution of such over at least a substantial portion of the target area. When different sets of voxels are formed in different voxel directions, the imaging member may construct cross-voxels each of which is defined as an intersecting or overlapping portion of such voxels.

Regardless of whether the movable member performs only the forward linear translation or the reciprocation, the imaging member can generate cross-voxels in the image domain. For example, during the linear translation, the imaging member can define a series of vertical voxels sequentially along the X-axis at each of the measurement locations using the output signals generated by the scanning units comprised of S₁-D₁-D₄-S₂ and S₁-D₂-D₃-S₂. After the linear translation is completed, the imaging member can also define a series of auxiliary horizontal voxels sequentially along the Y-axis. In other words, by assuming that the target area is at steady-state during the translation, the imaging member may regroup the wave sources and detectors to form auxiliary scanning units. For example, the wave source S₁ in positions A and D is grouped with the wave detector D₁ in positions B and C, thereby forming a scanning unit comprised of S₁ (in A)-D₁ (in B)-D₁ (in C)-S₁ (in D). In addition, other auxiliary scanning units may also be defined, e.g., S₁ (in A)-D₂ (in B)-D₂ (in C)-S₁ (in D), S₁ (in A)-D₂ (in B)-D₃ (in C)-S₂ (in D), S₁ (in A)-D₁ (in B)-D₃ (in C)-S₂ (in D), D₄ (in A)-S₂ (in B)-S₂ (in C)-D₄ (in D), etc. It is appreciated that the foregoing auxiliary scanning units all satisfy the symmetry requirements of the co-pending '972 application. In case such symmetry should not be required, the imaging member can further define non-symmetric scanning units, e.g., S₁ (in A)-D₁ (in B)-D₁ (in C)-S₂ (in D), etc

Once the foregoing horizontal and vertical voxels are defined in the image domain, the imaging member can also define cross-voxels by identifying vertical voxels intersecting with horizontal voxels. The imaging member can also calculate cross-voxel values from the voxel values of the intersecting vertical and horizontal voxels. Further details regarding the voxels and cross-voxels and their values are disclosed in a commonly assigned co-pending U.S. Application bearing Serial No. (N/A), entitled “Optical Imaging System for Direct Image Construction” and another commonly assigned co-pending U.S. Application bearing Serial No. (N/A) entitled “Optical Imaging System with Symmetric Optical Probe,” both of which have been filed on Feb. 5, 2001 and both of which are incorporated herein in their entirety by reference. It is noted that the cross-voxels may also be defined by moving the scanning unit or movable member along at least two non-parallel curvilinear paths. For example, in the embodiment shown in FIG. 4, the actuator member linearly translates the scanning unit in the X-direction. After the imaging member defines a series of vertical voxels sequentially along the X-direction, the actuator member then rotates the movable member about a pre-selected angle, e.g., 90° clockwise, and linearly translates the scanning unit or movable member upwardly. The imaging member then defines a series of horizontal voxels sequentially along the Y-direction. By identifying intersecting regions of the vertical and horizontal voxels in the target area, the imaging member constructs the cross-voxels in the image domain.

As discussed in FIG. 4, the stationary body may include one or more guiding tracks defining the path of the linear translation to generate high-precision movement of the movable member (or scanning unit). In the alternative, the stationary body may be provided with barriers along the bottom, sides, and top thereof so that movement of the scanning unit beyond the barriers are physically prevented, thereby confining movements of the movable member (or scanning unit) inside the region bordered by such barriers.

The actuator member may also be arranged to move the movable member at a pre-selected speed of translation. Alternatively, the actuating member may be provided with a control feature so that a physician may manipulate the movable member (or scanning unit) to move at a normal, pre-selected speed, to move along a desired guiding track, and/or to provide a recess between different movements of the movable member (or scanning unit) along different curvilinear paths. Other factors being equal, the speed of movement of the movable member (or scanning unit) generally adversely affects accuracy of the estimated values of the chromophore properties and the resolution of the final images thereof. Thus, the actuating member may be arranged to allow the physician to select an optimal speed of the scanning unit which may be determined based on several factors including, but not limited to, configuration of the scanning unit or movable member, resolution of the final images, frequency response of each sensor and/or other components of the optical imaging system, optical properties of the medium, size of the target area, and the like.

FIG. 5 is a schematic diagram of another exemplary scanning unit which is arranged to rotate or revolve according to the present invention. In this embodiment, the actuator member (not shown) rotates movable member 120 about a pre-selected center of rotation along a substantially arcuate path. For example, movable member 120 may have the center of rotation at its mid-point 129 such that rotations or revolutions thereof scan an arcuate or circular area having a radius which is substantially identical to one half length of scanning unit 125. Body 110 is also shaped and sized as an arc or circle to accommodate the shape and size of the arcuate or circular target area defined by scanning unit 125 and to minimize formation of the dead area or blind spot.

The actuator member may generate different types of rotations or revolutions of the scanning unit. For example, the actuator member may rotate the scanning unit about the center of rotation provided adjacent to one of the edges thereof. This embodiment results in an arcuate or circular target area having a diameter which is greater than the half-length of the scanning unit. Alternatively, the actuator member may generate two or more different movements, rendering the scanning unit define the scanned area composed of a combination of arcs and/or circles having different radii and/or different centers of rotation. In addition, the actuator member may manipulate the scanning unit to combine such arcuate or circular movements with linear translations discussed hereinabove. When it is desirable to provide such composite scanned areas, an optional controller may be provided to fine-control the movements of the movable member along the multiple, pre-selected curvilinear paths.

As described above, the actuator member may also be arranged to generate at least two movements of the movable member along at least two curvilinear paths or in at least two curvilinear directions. Such movements may be tailored to satisfy a pre-selected geometric arrangement therebetween. For example, at least a portion of a first curvilinear path (or direction) may be arranged to be substantially transverse to at least a portion of a second curvilinear path (or direction). Such curvilinear paths may also be arranged to be orthogonal to each other such as the axes of the Cartesian, cylindrical, and/or spherical coordinate systems. In particular, when the target area has a substantially polygonal shape, the actuator member may move the movable member along a first curvilinear path from a first side toward a second opposing side of the target area, then to move or reposition the movable member along a second curvilinear path from the second side to a third side thereof, and to move it along the third curvilinear path from the third side toward the first or other side of the polygon-shaped target area. Furthermore, one or more actuator members may provide different movements of the movable member, scanning unit, and/or sensors in different directions by operating each actuator member to generate a specific movement along a specific curvilinear path and/or by operating a single actuator member which can guide the movable member along different guiding tracks for different curvilinear paths. These embodiments, however, generally require meticulous control of the movable member and/or actuator member.

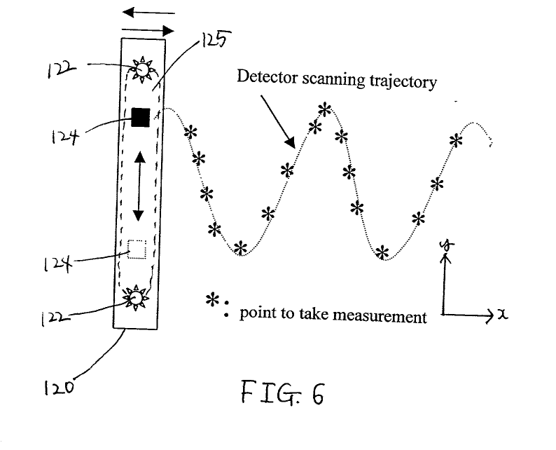

FIG. 6 is a schematic diagram of yet another exemplary scanning unit which is arranged for simultaneous X-translation and Y-reciprocation according to the present invention. The optical imaging system having this embodiment is substantially identical to those disclosed in FIGS. 4 and 5, except that the actuating member (not shown) generates a linear translation of movable member 120 along the X-axis while the same or an additional actuator member generates reciprocation of movable member 120 simultaneously along the Y-axis.

In operation, movable member 120 is disposed in a starting position such as one side of a housing (not shown), and stationary (or movable) body 110 is positioned on the target area. Wave sources 122 and detectors 124 are positioned on the medium to form optical coupling with a first region of the target area and activated to irradiate and detect electromagnetic waves. The actuator member translates movable member 120 along the X-axis while reciprocating movable member 120 along the Y-axis. Thus, movable member 120 (along with scanning unit 125) scans the target area along a sinusoidal path while the imaging member samples the foregoing output signals at desirable time intervals and/or pre-selected locations over the target area. Detailed configuration of the sinusoid such as its amplitude, frequency, and phase angle may be manipulated by controlling the speed of X-translation as well as that of Y-reciprocation. Once movable member 120 reaches the other side of the target area or the vicinity thereof, an operator may terminate the scanning process of the target area and manually move body 110 of optical imaging system 100 to the next adjacent target area of the medium for further scanning. Alternatively, the actuator member or an auxiliary motion generating member may be provided to mechanically translate and/or rotate body 110 and its scanning unit 125 to the next target area as well.

Signal-to-noise ratio of the output signal can be improved and resolution of the final images enhanced by repeating the foregoing scanning process over the target area. For example, the movable member may be moved back to the first region of the target area through the backward X-translation accompanied by the Y-reciprocation. The actuator member may move the movable member substantially along the similar or same sinusoidal path in the opposite direction and the imaging member may sample the output signals from the wave detectors at the similar or same measurement locations and/or at the similar or same sampling rate during the backward movement. By obtaining multiple output signals during the forward and backward movements and averaging such signals, the signal-to-noise ratio of the output signal can be dramatically improved. Alternatively, the actuator member may generate different sinusoidal paths and/or the imaging member may sample the output signals at different locations or sampling rates. Therefore, at least two different sets of voxels or measurement elements may be defined in the image domain during each of the forward and backward movements of the movable member. If preferred, the movable member may perform the Y-reciprocation while the movable member is moved at a slower speed or stalled at desired positions along the X-axis. Only after the entire height of the target area is scanned by the scanning unit, the movable member resumes the normal X-translation. This embodiment provides an advantage of allowing the smaller and shorter movable member to scan the entire target area.

The major advantage accomplished by the embodiment of FIG. 6 is that such optical imaging system only need to include a minimum number of the wave sources and/or detectors. Contrary to the embodiments shown in FIGS. 4 and 5 where the scanning units preferably have a characteristic dimension (e.g., height and radius, respectively) which is substantially identical to that of the target area, the optical imaging system of FIG. 6 defines the scanning unit with its height or width substantially less than that of the target area. By reciprocating the shorter scanning unit in the vertical direction, however, the scanning unit can cover the entire height of the target area. By translating such scanning unit along the horizontal direction, the scanning unit can scan the entire width of the target area as well Therefore, the foregoing optical imaging system may even be able to employ a single-probe-single-detector arrangement which may define the scanning unit having the scanning area only a tiny fraction of the target area.

The actuator member for generating two or more different movements along two or more curvilinear paths generally provides the voxels or measurement elements in two or more directions in the image domain. For example, the embodiment shown in FIG. 6 allows the imaging member to define the voxels not only along the X-axis but also along the Y-axis. That is, the imaging member can define more than one set of voxels in the direction which may be orthogonal to the path of the linear translation. By manipulating the speeds along both of X- and Y-axes and by synchronizing the sampling rates or sampling positions with such movements, the shape and size of the voxels and their arrangement may be readily manipulated as well. It is noted that the voxels obtained by two simultaneous movements of the movable member roughly correspond to the cross-voxels obtained by two sequential and/or non-parallel movements. This may be generalized to any movements of the movable member along any curvilinear paths. For example, an actuator member may be arranged to rotate the movable member while linearly translating or reciprocating it along the radial direction. This arrangement yields a series of spiral layers in the radial direction, where each turn of a spiral layer may contain multiple arcuate voxels. By maintaining the rotational speed greater than the radial translational speed, the shapes of the spiral layers approach concentric shells each of which includes multiple arcuate voxels therein. Further details of such voxels are provided in the foregoing commonly assigned co-pending U.S. Applications entitled “Optical Imaging System for Direct Image Construction” and “Optical Imaging System with Symmetric Optical Probe,” both of which have been filed on Feb. 5, 2001 and both if which are incorporated herein in their entirety by reference.

In yet another aspect of the present invention, an optical imaging system directly generates the foregoing cross-voxels by incorporating at least one of the foregoing wave sources and/or detectors into a movable component of the optical imaging system and by incorporating at least one of the foregoing wave sources and/or detectors into a stationary component thereof.

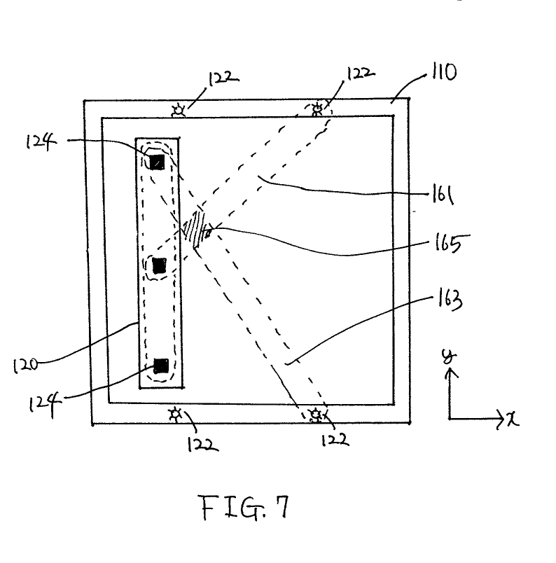

FIG. 7 is a schematic diagram of another exemplary scanning unit according to the present invention, where wave sources 122 are disposed along one side of a stationary body 110, whereas wave detectors 124 are included in a movable body 120 which is linearly translated by an actuator member (not shown) along the X-axis of target area. Accordingly, wave sources 122 and detectors 124 define scanning units 161, 163 which are elongated at angles with respect to the linear movement path of movable member 120 and which change their configuration (such as their sizes, shapes, angles, and the like) during the movement of movable member 120.

In operation, stationary body 110 is placed on a target area of the medium and movable member 120 is positioned in its starting position such that wave sources 122 form stationary optical coupling with the target area and wave detectors 124 form movable optical coupling with a first region of the target area. Wave sources 122 and detectors 124 are activated to irradiate and detect electromagnetic waves. The actuator member translates movable member 120 and its wave detectors 124 from the first region to an adjacent region of the target area along a linear path which generally corresponds to the X-axis of the target area. Depending upon the sampling rate of the imaging member (not shown), each pair of wave source 122 and detector 124 forms an angled voxel 161, 163 formed at varying angles with respect to the linear translation path of movable member 120 (or the X-axis of the target area). Wave detectors 124 generate output signals which are generally obtained by averaging the output signals over an entire area of each elongated, angled voxel 161, 163. The imaging member receives such output signals and determines voxel values for each elongated voxel 161, 163. Because wave sources 122 and detectors 124 define multiple angled voxels 161, 163 which intersect each other in the target area, the imaging member can define cross-voxels 165 and calculate the cross-voxel values. Once movable member 120 reaches the opposing side of the target area or the vicinity thereof, the scanning process may be terminated and a new scanning process may be initiated by moving body 110 to the next target area. In the alternative, the actuator member may repeat the foregoing scanning process of the same target area along the identical or different curvilinear paths.

It is noted that the scanning unit of FIG. 7 generally defines angled voxels 161, 163 which change their shapes and sizes during the movement of movable member 120, because the geometric arrangements between stationary wave sources 122 and movable wave detectors 124 vary depending on the position of movable member 120 over the target area. Such voxels 161, 163 may require more complicated analytic or numerical schemes for obtaining solutions of the wave equations applied to such stationary wave sources 122 and movable wave detectors 124 and, therefore, generally not preferred to the ones which maintain substantially identical shapes and sizes. However, differences in the shapes and sizes among the angled voxels and their cross-voxels may also be compensated by various arrangements, e.g., by synchronizing the actuator member and imaging member so that the signal or data may be sampled at pre-selected positions of the target area, thereby defining the voxels having pre-determined configurations. Configuration of the cross-voxels and distribution pattern thereof may also be controlled by manipulating geometric arrangements between the wave sources and detectors, by varying the speed of movement of the wave detectors, by manipulating the contour of the curvilinear movement path of the scanning unit, and so on. Therefore, it is generally a matter of choice of one skilled in the art to find the optimum arrangement for the scanning unit, actuator member, and/or imaging member in the foregoing embodiment. If preferred, at least one wave source may be disposed in the movable member and/or at least one wave detector may be implemented to the stationary body. The foregoing source-detector arrangement can also be reversed, i.e., all of the wave sources are disposed at the movable member, while all of the wave detectors are disposed at the stationary body.

As described above, the accuracy of the output signal and resolution of the images may be enhanced by repeating the scanning process or performing different scanning processes over the same target area. Multiple sets of the cross-voxels may also be provided by, e.g., adjusting the grouping or sampling pattern of the sensors and/or by manipulating the actuator member to vary the path and/or speed of movements of the movable member. Furthermore, the embodiment of FIG. 7 may also incorporate a minimum number of wave sources and detectors, and their scanning units may have the height and width substantially less that those of the target area.

It is noted that the foregoing optical imaging systems and optical probes of the present invention can generate the images of two- and/or three-dimensional distribution of the chromophores or their properties on a substantially real time basis. Contrary to prior art optical technology requiring complicated and time-consuming image reconstruction process, the foregoing optical imaging system may generate such images directly from the estimated chromophore properties determined from the cross-voxel values of the foregoing cross-voxels. For examples, the optical imaging systems of FIGS. 4 through 7 may provide the images on a real time basis regardless of the size of the target area, number of wave sources and detectors, and detailed configuration of the curvilinear paths along which the movable member travels. The foregoing optical imaging systems may be readily equipped to provide variable resolutions of the images. Contrary to the prior art counterparts all of which require complicated readjustment of the equipment, the operator can readily adjust or vary the image resolution by, e.g., manipulating the sampling rate, speed of movement of the movable member, grouping or sampling pattern of the wave sources and detectors, and so on.

In another aspect of the invention, an optical imaging system may include a movable body and movable member to generate images of a target area of a physiological medium by moving the movable member within the target area as well as by moving the movable body over different target areas of the medium.

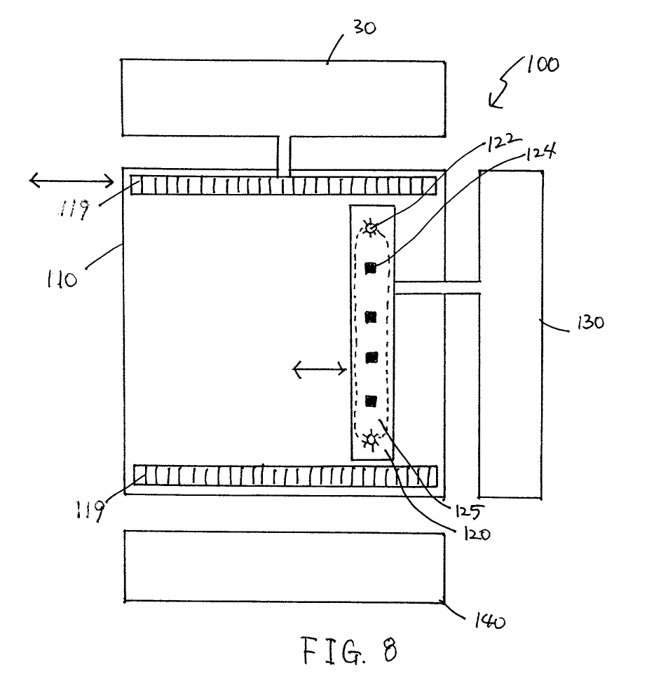

FIG. 8 is a schematic diagram of another optical imaging system according to he present invention. Such optical imaging system 100 includes a movable body 110, at east one movable member 120, an actuator member 130, and an imaging member 140. Movable body 110 is shaped and sized to cover at least a substantial portion of the target area of the medium and preferably arranged to enclose therein at least a portion of movable member 120. Movable body 110 includes at least one moving unit or displacement unit 119 capable of moving movable body 110 over different target areas of the medium. Examples of such displacement units 119 may include, but not limited to, wheels, rollers, caterpillar tracks, etc. Movable member 120 includes at least one of the foregoing wave sources 122 and at least one of the foregoing wave detectors 124 arranged according to the configuration described hereinabove. Actuator member 130 operationally couples with both of movable body 110 and movable member 120 to generate at least one primary movement of movable body 110 along at least one primary curvilinear path and at least one secondary movement of movable member 120 along at least one secondary curvilinear path. Actuator member 130 generates curvilinear translations, rotations, revolutions or reciprocations of movable body 110 and/or movable member 120 simultaneously or sequentially.

In operation, movable body 110 and movable member 120 are positioned in their starting positions. Movable body 110 is placed on the medium so that scanning unit 125 is positioned in a first region of the target area of the medium. Wave sources 122 and wave detectors 124 are then activated and actuator member 130 translates movable member 120 substantially linearly to adjacent regions of the target area. Once movable member 120 reaches the other side of the target area, the scanning process may be terminated or movable member 120 may be moved back to the first region of the target area while continuing the scanning process. After movable member 120 finishes scanning of all regions of the target area, moving unit 119 is activated to move the optical probe and/or entire optical imaging system to the second target area of the medium.

It is appreciated that an optional guiding member may be provided on the medium so that the movable member may travel thereon across different regions of a target area and/or different target areas of the medium. Such guiding member may preferably be made of flexible material or may have a structure such that its shape can conform to surface contours of different regions and/or target areas of the medium. For example, a ring-shaped guiding member may be installed around a head or a base of a human breast. The movable member movably engages with the guiding member and moves therealong while scanning the head or breast. By allowing the movable member to travel along the curvilinear path with pre-selected spatial coordinates at a preferred speed, the optical imaging system can readily provide two- or three-dimensional distribution of the chromophore properties around the head or the breast of the test subject. In addition, by moving the optical probe or entire optical imaging system along the guiding member which is positioned in the pre-selected position with known coordinates, the optical imaging system of the present invention can readily construct the three-dimensional images of the distribution of chromophore property from the two-dimensional images of the distribution thereof without relying on any image markers conventionally required by the prior art optical imaging technology.

Although the above disclosure of the present invention is mainly directed to provide images of spatial distribution of the chromophore property, the present invention may be applied to generate images of temporal distribution thereof. As briefly discussed above, the scanning unit of the movable member may be arranged to scan the substantially same region over a time interval. By obtaining the differences in the output signals detected at different time intervals in the same target area, the imaging member calculates temporal changes in the chromophore properties of the target area and generates the images of the temporal distribution of such properties. Alternatively, the temporal distribution may be determined and its images may be provided from spatial distributions of such chromophore property determined in different time frames. For example, the movable member and its scanning unit may repeat scanning of the target area and calculate the temporal distribution pattern of the chromophore property in each region of the target area. It is noted that the temporal changes and their distribution usually relate to relative changes in the property of the chromophores. However, once the absolute values of such chromophore properties are determined in any reference time frame, preceding or subsequent changes in such properties can be readily converted to the absolute values thereof and vice versa.

It is noted that the foregoing optical imaging systems, optical probes thereof, and methods therefor of the present invention may provide values for the temporal changes in blood or water volume in the target area of the medium. In an exemplary embodiment for calculating such temporal changes in blood volume in a target area of a human subject, the concentration of oxygenated hemoglobin, [HbO], and that of deoxygenated hemoglobin, [Hb], are calculated by a set of equations (1a) and (1b) or by another set of equations (2a) and (2b). Once [Hb] and [HbO] are obtained, the sum (i.e., total hemoglobin concentration, [HbT], which is the sum of [Hb] and [HbO]) is also calculated. By obtaining the output signals from the wave detectors positioned in the same target area over time, changes in the total hemoglobin concentration is obtained. By assuming that hematocrit of blood (i.e., the volume percentage of the red blood cells in blood) flowing in and out of the target area is maintained at a constant level over time, temporal changes in the blood volume in the target area are directly calculated in terms of temporal changes in [HbT] in the target area. In the alternative, temporal changes in [Hb] and [HbO] may be calculated from the equations (6a) and (6b) and temporal changes in [HbT] is then obtained as the sum of changes in [Hb] and [HbO] in the target area.

It is also appreciated that the optical imaging systems, optical probes thereof, and methods therefor of the present invention may be applied to obtain the images of three-dimensional distribution of the chromophore properties in a target area of the medium. As discussed above, electromagnetic waves are irradiated by the wave sources and transmitted through a target volume of the medium which is defined by a target area and a pre-selected depth (or thickness) into the medium. Therefore, a set of wave equations can be formulated for such three-dimensional target volume, and the output signals generated by the wave detectors are provided to the imaging member which then solves the wave equations with relevant initial and/or boundary conditions, where such solutions from the wave equations represent the three-dimensional distribution of the chromophore properties in the target volume of the medium. To maintain the pre-selected resolution of the images, the optical imaging systems and/or probes thereof preferably include enough number of wave sources and detectors arranged to define a greater number of voxels in the target volume. Suppose an exemplary optical imaging system includes two wave sources and four wave detectors and generates two-dimensional images of a target area at a pre-selected resolution. When a target volume is defined to have the same target area and a pre-selected thickness including N two-dimensional layers stacked one over the other, such an optical imaging system may probably be required to include about 2N wave sources and 4N wave detectors in order to maintain the same resolution for each two-dimensional layer. The number of requisite wave sources and detectors may be reduced, however, by generating enough movements of two wave sources and four wave detectors over the target area, preferably in multiple different curvilinear directions. However, the required number of wave sources and detectors is generally inversely proportional to the number and/or complexity of the movements of the movable member (or scanning unit) or to the sampling rate of the imaging member. Thus, the optical imaging system may require fewer number of wave sources and detectors by arranging the actuator member to generate more movements of the scanning unit and/or by arranging the imaging member to sample output signals at a higher rate. It is appreciated, however, that resolution of images from any optical imaging system is inherently limited by the average “free walk distance” of photons in the physiological medium which is typically about 1 mm. In addition, due to sensitivity limitation and/or electronic and mechanical noise inherent in almost any optical imaging system, the best-attainable resolution of the optical imaging system may be in the range of a few millimeters or about 1 mm to 5 mm for now. Accordingly, the foregoing voxels and cross-voxels which have dimensions less than 1 mm to 5 mm or, more particularly, about 1 mm may not necessarily enhance resolution of the final images.

The foregoing optical imaging systems and optical probes of the invention can also be used in both non-invasive and invasive procedures. For example, such optical probes may be non-invasively disposed on the target area of an external surface of the test subject. In the alternative, a miniaturized optical probe may be implemented to a tip of a catheter which is invasively disposed on an internal target area of the test subject as well. The foregoing optical imaging systems may be employed to determine intensive properties of the chromophores such as concentration, sum of concentrations, and/or ratios of such concentrations. The foregoing optical imaging system may be used to estimate extensive chromophore properties such as volume, mass, weight, volumetric flow rate, and mass flow rate thereof.

It is appreciated that the foregoing optical imaging systems, optical probes thereof, and methods therefor may be readily adjusted to provide images of distribution of different chromophores or properties thereof. Because different chromophores generally respond to electromagnetic waves having different wavelengths, the wave sources of such optical imaging systems and probes may be manipulated to irradiate electromagnetic waves interacting with pre-selected chromophores. For example, the near-infrared waves having wavelengths between 600 nm and 1,000 nm, e.g., about 690 nm and 830 nm are suitable to measure the distribution pattern of the hemoglobins and their property. However, the near-infrared waves having wavelengths between 800 nm and 1,000 nm, e.g., about 900 nm, can also be used to measure the distribution pattern of water in the medium. Selection of an optimal wavelength for detecting a particular chromophore generally depends on optical absorption and/or scattering properties of the chromophore, operational characteristics of the wave sources and/or detectors, and the like.

The foregoing optical imaging systems, optical probes, and methods of the present invention may be clinically applied to detect tumors or stroke conditions in human breasts, brains, and any other areas of the human body where the foregoing optical imaging methods such as diffuse optical tomography is applicable. The foregoing optical imaging systems and methods may also be applied to assess blood flow into and out of transplanted organs or extremities and/or autografted or allografted body parts or tissues. The foregoing optical imaging systems and methods may be arranged to substitute, e.g., ultrasonogram, X-rays, EEG, and laser-acoustic diagnostic. Furthermore, such optical imaging systems and methods may be modified to be applicable to various physiological media with complicated photon diffusion phenomena and/or with non-flat external surface.

It is noted that the optical imaging systems, optical probes, and methods of the present invention may incorporate or be applied to related inventions and embodiments disclosed in the commonly assigned co-pending U.S. Application bearing Serial No. (N/A), entitled “Optical Imaging System for Direct Image Construction,” another commonly assigned co-pending U.S. Application bearing Serial No. (N/A), entitled “Self-Calibrating Optical Imaging System,” and yet another commonly assigned co-pending U.S. Application bearing Serial No. (N/A), entitled “Optical Imaging System with Symmetric Optical Probe,” all of which have been filed on Feb. 5, 2001 and all of which are incorporated herein in their entirety by reference.

Following example describes an exemplary optical imaging system, optical probe, and methods thereof according to the present invention. The results indicated that the foregoing exemplary optical imaging system provided reliable and accurate images of two-dimensional distribution of the blood volume and the oxygen saturation in the target areas of the human breast tissues.

### EXAMPLE

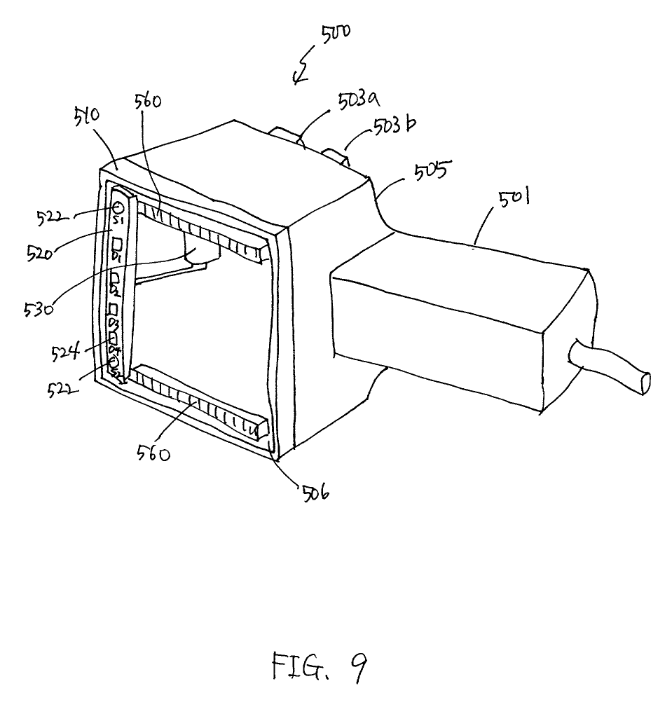

An exemplary optical imaging system 500 was constructed to obtain images of two-dimensional distribution of blood volume and oxygen saturation in target areas of female human breasts. FIG. 9 is a schematic diagram of a prototype optical imaging system according to the present invention.

Prototype optical imaging system 500 typically included a handle 501 and a main housing 505. Handle 501 was made of poly-vinylchloride (PVC) and acrylic stock, and provided with two control switches 503a, 503b for controlling operations of various components of system 500. Main housing 505 included a body 510, a movable member 520, an actuator member 530, an imaging member (not shown), and a pair of guiding tracks 560.

Body 510 was shaped as a substantially square block (3.075″×2.8″×2.63″) and provided with barriers along its sides. Body 510 was arranged to movably couple with rectangular movable member 520 (1.5″×2.8″×1.05″) designed to linearly translate along a path substantially parallel with one side of body 510.

Movable member 520 included two wave sources 522, S₁ and S₂, each of which was capable of irradiating electromagnetic waves having different wavelengths. In particular, each wave source 522 included two laser diodes, HL8325G and HL6738MG (ThorLabs, Inc., Newton, N.J.), where each laser diode irradiated the electromagnetic waves with wavelengths of 690 nm and 830 nm, respectively. Movable member 520 also included four identical wave detectors 524 such as photo-diodes D₁, D₂, D₃, and D₄, (OPT202, Burr-Brown, Tucson, Ariz.) which were interposed substantially linearly between wave sources 522. Wave sources 522 and detectors 524 were spaced at identical distances such that the foregoing sensors 522, 524 satisfy the foregoing symmetry requirements of the co-pending '972 application.

Actuator member 530 included a high-resolution linear-actuating-type stepper motor (Model 26000, Haydon Switch and Instrument, Inc., Waterbury, Conn.) and a motor controller (Spectrum PN 42103, Haydon Switch and Instrument, Inc.). Actuator member 530 was mounted on body 510 and engaged with movable member 520 so as to linearly translate movable member 520 along guiding tracks 560 fixedly positioned along the linear path. A pair of precision guides (Model 6725K11, McMaster-Carr Supply, Santa Fe Springs, Calif.) was used as guiding tracks 560.

The imaging member was provided inside handle 501 and included a data acquisition card (DAQCARD 1200, National Instruments, Austin, Tex.). Main housing 505 was made of acrylic stocks and constructed to open at its front face. Perspex Non-Glare Acrylic Sheet (Liard Plastics, Santa Clara, Calif.) was installed on a front face 506 of housing 505 and used as a protective screen to protect wave sources 522 and detectors 524 from mechanical damages.

In operation, movable member 520 was positioned in its starting position, i.e., the far-left side of body 505. An operator turned on the main power of system 500 and tuned wave sources 522 and detectors 524 by running scanning system software. A breast of a human subject was prepped and body 505 was positioned on the breast so that sensors 522, 524 of movable member 520 were placed in a first target area of the breast and formed appropriate optical coupling therewith. The first target area was scanned by clicking one control switch 503a on handle 501. Actuator member 530 translated movable member 520 linearly along one side of body 510 along guide tracks 560.

Wave sources 522 were synchronized to ignite their laser diodes in a pre-selected sequence. For example, a first laser diode of the wave source, S₁, was arranged to irradiate electromagnetic waves of wavelength 690 nm and wave detectors 524 detected the waves and generated a first set of output signals in response thereto. During the foregoing irradiation and detection period which generally lasted about 1 msec (with duty cycle from 1:10 to 1:1,000), all other laser diodes were turned off to minimize interference noises. After completing the irradiation and detection, the first laser diode of the wave source, S₁, was turned off and the first laser diode of the wave source, S₂, was turned on to irradiate electromagnetic waves of the same wavelength, 690 nm. Wave detectors 524 detected the waves and generated a second set of output signals accordingly. Other laser diodes were maintained at off positions during the foregoing irradiation and detection period as well. Similar procedures were repeated to the second laser diodes of the wave sources, S₁ and S₂, where both second laser diodes were arranged to sequentially irradiate the electromagnetic waves having wavelengths 830 nm.

The imaging member was also synchronized with wave sources 522 and detectors 524 and sampled the foregoing sets of output signals in a pre-selected sampling rate. In particular, the imaging member was arranged to process such output signals by defining a first and second scanning units, where the first scanning unit was comprised of the wave sources, S₁ and S₂, and the wave detectors, D₁ and D₄, and the second scanning unit was made up of the wave sources, S₁ and S₂, and the wave detectors, D₂ and D₃. Both of the first and second scanning units had the source-detector arrangement which satisfied the symmetry requirements of the co-pending '972 application. Therefore, concentrations of the oxygenated and deoxygenated hemoglobins were obtained by the equations (1a) to (1e), and the oxygen saturation, SO₂, by the equation (1e). Furthermore, relative values of blood volume (i.e., temporal changes thereof) was calculated by assessing the changes in hematocrit in the target areas as discussed above.

Actuator member 530 was also synchronized with the foregoing irradiation and detection procedures so that wave sources 522 and detectors 524 scanned the entire target area (i.e., irradiating electromagnetic waves thereinto, detecting such therefrom, and generating the output signals) before they were moved to the next adjacent region of the target area by actuator member 530. When movable member 520 reached the opposing end of body 510, actuator member 530 translated movable member 520 linearly to its starting position. The foregoing irradiation and detection procedures were repeated during such backward linear movement of movable member 520 as well. After the scanning procedure was completed, the operator pushed the other control switch 503b to send a signal to the imaging member which started image construction process and provided two-dimensional images of spatial distribution of the oxygen saturation in the target area and the temporal changes in the blood volume therein.

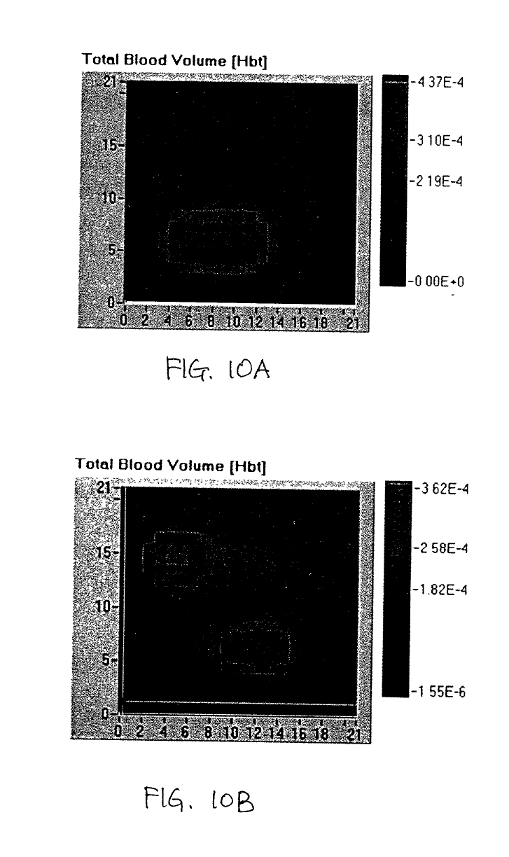

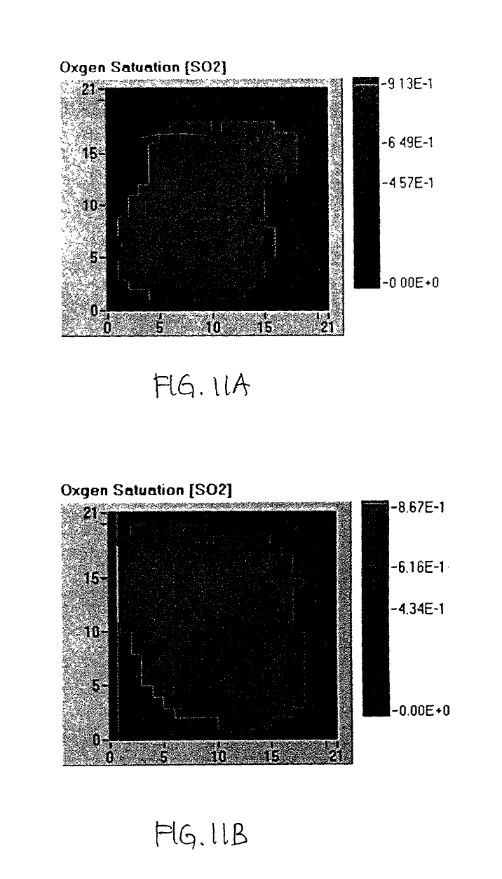

FIGS. 10A and 10B are two-dimensional images of blood volume in normal and abnormal breast tissues, respectively, both measured by the optical imaging system of FIG. 9. In addition, FIGS. 11A and 11B are two-dimensional images of oxygen saturation in normal and abnormal breast tissues, respectively, both measured by the optical imaging system of FIG. 9 according to the present invention. As shown in the figures, the optical imaging system provided that normal tissues had the higher oxygen saturation (e.g., over 70%) in the area with the maximum blood volume. However, the higher oxygen saturation in the corresponding area of the abnormal tissues was as low as 60%.

It is to be understood that, while various embodiments of the invention has been described in conjunction with the detailed description thereof, the foregoing is only intended to illustrate and not to limit the scope of the invention, which is defined by the scope of the appended claims. Other embodiments, aspects, advantages, and modifications are within the scope of the following claims.

What is claimed is:

1. An optical imaging system configured to generate images of a target area of a physiological medium, said images representing distribution of properties of hemoglobins in said target area, said system comprising:
at least one movable member having at least one wave source and at least one wave detector, said at least one wave source configured to irradiate near-infrared electromagnetic waves into said target area and said at least one wave detector configured to detect said near-infrared electromagnetic waves from said target area of said and to generate output signal in response thereto; and
an actuator configured to operationally couple with said movable member and to generate at least one movement of said movable member with respect to said target area along at least one curvilinear path.

2. The system of claim 1 wherein said distribution is at least one of two-and three-dimensional distribution of said hemoglobins.

3. The system of claim 1 wherein said distribution is at least one of spatial and temporal distribution of said hemoglobins.

4. The system of claim 1 wherein said properties are absolute values of concentration of said hemoglobins.

5. The system of claim 1 wherein said properties are relative values of said hemoglobins, said values representing at least one of spatial and temporal changes in said hemoglobins.

6. The system of claim 1 wherein said properties include at least one of concentration of said hemoglobins, a sum of at least two concentrations thereof, and a ratio thereof.

7. The system of claim 1 wherein said properties include at least one of volume, mass, weight, volumetric flow rate, and mass flow rate thereof.

8. The system of claim 1 wherein said properties at least one of concentration of oxygenated hemoglobin, concentration of deoxygenated hemoglobin, and oxygen saturation defined as a ratio of said concentration of oxygenated hemoglobin to a sum of said concentrations of oxygenated and deoxygenated hemoglobins.

9. The system of claim 1 wherein said at least one wave source is configured to irradiate near-infrared electromagnetic waves having different wave characteristics.

10. The system of claim 1 wherein said at least one wave detector is configured to detect near-infrared electromagnetic waves having different wave characteristics.

11. The system of claim 1 wherein said movement includes at least one of curvilinear translation, reciprocation, rotation, revolution, and a combination thereof.

12. The system of claim 1 wherein said actuator is configured to generate at least one movement at a constant speed.

13. The system of claim 1 wherein said actuator is configured to generate at least one movement at a variable speed.

14. The system of claim 1 wherein said movement has temporal characteristics which are at least one of an impulse, step, pulse, pulse train, sinusoid, and a combination thereof.

15. The system of claim 1 wherein said movement is at least one of periodic, aperiodic, and intermittent.

16. The system of claim 1 wherein the movable member has a longitudinal axis and said at least one wave source and at least one detector are disposed along said longitudinal axis and are configured to form a scanning unit elongated along said longitudinal axis, said scanning unit configured to move with said movable member and to define therearound a scanning area in which said wave detector can detect near-infrared electromagnetic waves transmitted from said target area.

17. The system of claim 16 wherein said scanning area is smaller than said target area.

18. The system of claim 16 wherein at least a portion of said curvilinear path of said movement is substantially orthogonal to said longitudinal axis.

19. The system of claim 16 wherein at least a portion of said curvilinear path of said movement substantially parallel to said longitudinal axis.

20. The system of claim 16 wherein said movable member includes at least two wave detectors which are disposed substantially along said longitudinal axis.

21. The system of claim 20 wherein said movable member includes at least two wave sources disposed substantially along said longitudinal axis.

22. The system of claim 21 wherein at least two wave detectors are interposed between at least two wave sources.

23. The system of claim 22 wherein a first near-distance between a first wave source and a first wave detector is substantially similar to a second near-distance between a second wave source and a second wave detector, and wherein a first far-distance between said first wave source and said second wave detector is substantially similar to a second far-distance between said second wave source and said first wave detector.

24. The system of claim 21 wherein at least two wave sources are interposed between at least two wave detectors.

25. The system of claim 20 wherein said movable member includes at least two wave sources, a first wave source disposed on one side across said longitudinal axis and a second wave source disposed on the other side across said longitudinal axis.

26. The system of claim 25 wherein said first and second wave sources are configured to be disposed substantially symmetrically with respect to said longitudinal axis.

27. The system of claim 1 wherein said actuator is configured to generate at least two movements of said movable member along at least two curvilinear paths.

28. The system of claim 27 wherein said actuator is configured to generate sequential movements sequentially.

29. The system of claim 27 wherein said actuator is configured to generate at least a portion of a first movement and at least a portion of a second movement simultaneously.

30. The system of claim 27 wherein at least a portion of a first curvilinear path is substantially orthogonal to at least a portion of a second curvilinear path.

31. The system of claim 30 wherein at least two curvilinear paths are orthogonal axes of one of the Cartesian, cylindrical, and spherical coordinate systems.

32. The system of claim 1 wherein said actuator member is configured to sequentially generate at least two movements of said movable member, a first movement starting from a first portion of said target area toward a second portion thereof and a second movement starting from said second portion toward said first portion of said target area.

33. The system of claim 1 wherein said actuator member is configured to sequentially generate at least three movements of said movable member, a first movement starting from a first side of said target area toward a second side thereof, a second movement starting from said second side to a third side of said target area, and a third movement starting from said third side toward a fourth side of said target area.

34. The system of claim 33 wherein said first and third movements are substantially linear translations and said second movement is substantially rotation.

35. The system of claim 33 wherein said target area has a shape of a rectangle, wherein said first and second sides are a first pair of opposing sides of said rectangle and wherein said third and fourth sides are a second pair of opposing sides of said rectangle.

36. The system of claim 27 wherein said actuator is configured to simultaneously generate a first and second movements of said movable member along a first and second curvilinear paths, respectively, at least a portion of said first curvilinear path configured to be substantially orthogonal to at least a portion of said second curvilinear path.

37. The system of claim 36 wherein one of said first and second movements is substantially linear translation and the other of said first and second movements is substantially reciprocation.

38. The system of claim 1 wherein said at least one wave source and at least one detector are non-invasively disposed over said target area of said medium.

39. The system of claim 1 wherein said at least one wave source and at least one detector are configured to be invasively positioned over said target area disposed inside said medium.

40. An optical imaging system configured to generate images of a target area of a physiological medium, said images representing distribution of properties of hemoglobins in said target area, said system comprising:
at least one sensor assembly having a wave source and a wave detector, said wave source capable of irradiating near-infrared electromagnetic waves into said medium, and said wave detector configured to detect said near-infrared electromagnetic waves from said medium and to generate output signal in response thereto;
a body configured to support said sensor assembly; and
an actuator configured to operationally couple with at least one of said sensor assembly and body and to generate at least one movement of at least one of said sensor assembly and body with respect to said target area along a curvilinear path.

41. The system of claim 40 wherein said movement includes at least one of curvilinear translation, reciprocation, rotation, revolution, and a combination thereof.

42. The system of claim 40 wherein said sensor assembly fixedly couples with said body, said actuator configured to move both of said sensor assembly and body with respect to said target area.

43. The system of claim 40 wherein said sensor assembly movably couples with said body, said actuator member configured to move said sensor assembly with respect to said body and target area.

44. The system of claim 40 wherein said sensor assembly movably couples with said body, said actuator member configured to generate a first movement of said sensor assembly with respect to said body and target area and to generate a second movement of said body with respect to said target area.

45. The system of claim 44 wherein said actuator is configured to generate at least a portion of said first movement of said sensor assembly simultaneously with at least a portion of said second movement of said body.

46. The system of claim 44 wherein said actuator member is configured to generate said first and second movements sequentially.

47. The system of claim 40 wherein said body includes a moving unit configured to move both of said sensor assembly and body from said target area to another target area of said medium.

48. An optical imaging system configured to generate images of a target area of a physiological medium, said images representing distribution of properties of hemoglobins in said target area, said system having one or more wave sources configured to irradiate near-infrared electromagnetic waves into said medium and one or more wave detectors configured to detect said near-infrared electromagnetic waves and to generate output signal in response thereto, said system comprising:
at least one portable probe including at least one movable member and an actuator member, said movable member including at least one of said wave source and at least one of said wave detector, and said actuator member configured to operationally couple with said movable member and to generate at least one movement of said movable member along at least one curvilinear path; and
a console including an imaging member configured to receive said output signal, to determine said distribution of said properties of hemoglobins and to generate said images of said distribution.

49. The system of claim 48 further comprising:
a connector member configured to provide at least one of electrical communication, optical communication, electric power transmission, mechanical power transmission, and data transmission between said portable probe and console.

50. The system of claim 49 wherein said connector member includes at least one fiber optic article.

51. The system of claim 48 wherein said portable probe includes a rechargeable power source and forms an article detachable from said console.

52. The system of claim 51 wherein said portable probe is configured to communicate with said console telemetrically.

53. The system of claim 51 wherein said portable probe includes a memory member capable of storing at least one of said output signal, a signal representing said distribution, and a signal representing said images.

54. An optical imaging system configured to generate images of a target area of a physiological medium, said images representing distribution of properties of hemoglobins in said target area, said optical imaging system comprising:
at least one wave source configured to irradiate near-infrared electromagnetic waves into said medium;
at least one wave detector configured to generate output signal in response to said near-infrared electromagnetic waves detected thereby; and
at least one optical probe including at least one movable member and at least one actuator member, said movable member including at least one of said wave source and detector, and said actuator member configured to operationally couple with said movable member and to generate at least one movement of said movable member along at least one curvilinear path.

55. The system of claim 54 further comprising:
a console operationally coupling with said optical probe and including an Imaging member configured to receive said output signal, to determine said distribution of said properties of said hemoglobins from a set of solutions of a plurality of wave equations applied to said wave source and detector, and to generate said images of said distribution.

56. An optical imaging system configured to generate images of a target area of a physiological medium, said images representing distribution of properties of hemoglobins in said target area, said optical imaging system comprising:
at least two wave sources configured to emit near-infrared electromagnetic waves into said medium; and
at least two wave detectors configured to generate output signal in response to said near-infrared electromagnetic waves detected thereby,
wherein at least two of said wave sources and at least two of said wave detectors are disposed substantially along a straight line.

57. The system of claim 56 further comprising:
an actuator member configured to generate movement of at least one of said wave sources and detectors.

58. The system of claim 57 wherein said actuator member is configured to move all of said wave sources and detectors disposed substantially linearly along said line.

59. The system of claim 57 wherein said movement includes at least one of curvilinear translation, reciprocation, rotation, revolution, and a combination thereof.

60. A method for generating images of a target area of a physiological medium by an optical imaging system, said images representing two- or three-dimensional distribution of properties of hemoglobins in said target area, wherein said optical imaging system includes at least one wave source, at least one wave detector, a movable member, and an actuator member, said wave source configured to emit near-infrared electromagnetic waves into said target area of said medium, said wave detector configured to generate output signal in response to said near-infrared electromagnetic waves detected thereby, said movable member having a longitudinal axis and configured to include at least one of said wave source and detector, and said actuator member operationally coupling with said movable member, wherein said wave source and detector are configured to form a scanning unit elongated along said longitudinal axis of said movable member and defining a scanning area therearound, and wherein said actuator member operationally couples with said movable member and is configured to generate at least one movement of said movable member along at least one curvilinear path, said method comprising:
positioning said movable member in a first region of said target area of said medium;
scanning said first region by irradiating said near-infrared electromagnetic waves thereinto by said wave source and by obtaining said output signal therefrom by said wave detector; and
manipulating said actuator member to generate said movement of said movable member from said first region to a second region of said target area along at least one curvilinear path.

61. The method of claim 60 further comprising:
repositioning said movable member sequentially in a plurality of target areas of said medium; and
repeating said scanning and manipulating steps in each of said target areas.

62. The method of claim 60 further comprising:
determining said distribution of said properties of said hemoglobins in said target area; and
obtaining said images representing said distribution in said target area.

63. The method of claim 60 wherein said positioning comprises at least one of:
forming optical coupling between said medium and said wave source and between said medium and said wave detector; and
maintaining at least a portion of said optical couplings during said movement of said movable member.

64. The method of claim 60 wherein said manipulating comprises one of:
moving said movable member at one constant speed; and
moving said movable member at speeds varying with respect to at least one of time and position of said target area.

65. The method of claim 60 wherein said manipulating comprises at least one of:
moving said movable member along said curvilinear path which is at least substantially orthogonal to said longitudinal axis of said movable member;
moving said movable member along said curvilinear path which is at least substantially parallel with said longitudinal axis; and
moving said movable member along said curvilinear path disposed at a pre-selected angle with respect to said longitudinal axis.

66. The method of claim 60 wherein said manipulating comprises at least one of:
linearly translating said movable member along at least one linear path;
translating said movable member along at least one curvilinear path;
rotating said movable member about at least one center of rotation about a pre-selected angle along at least one curved path;
revolving said movable member about at least one center of rotation for a pre-selected number of turns along at least one curved path; and
reciprocating said movable member along at least one curvilinear path.

67. The method of claim 60 wherein said manipulating comprises:
generating at least two movements of said movable member along at least two curvilinear paths.

68. The method of claim 67 wherein said generating comprises:
moving said movable member along at least two curvilinear paths in at least one of a simultaneous, sequential, and intermittent mode.

69. A method for generating images of a target area of a physiological medium by an optical imaging system, said images representing two- or three-dimensional distribution of properties of hemoglobins in said target area, wherein said optical imaging system includes a sensor assembly, a body, and an actuator member, said sensor assembly having at least one wave source configured to irradiate near-infrared electromagnetic waves to said medium and at least one wave detector configured to generate output signal in response to said near-infrared electromagnetic waves detected thereby, said body configured to support at least a portion of said sensor assembly, and said actuator member operationally coupling with at least one of said sensor assembly and said body and configured to generate at least one movement of at least one of said sensor assembly and said body, said method comprising:
positioning said sensor assembly in a first region of said target area of said medium;
scanning said first region with said sensor assembly by irradiating said near-infrared electromagnetic waves into said first region of said medium and by generating said output signal therefrom; and
manipulating said actuator member to generate said movement of at least one of said sensor assembly and said body from said first region toward a second region of said target area of said medium along at least one curvilinear path.

70. The method of claim 69 further comprising:
fixedly coupling said sensor assembly with said body; and
moving said body during said movement.

71. The method of claim 69 further comprising:
movably coupling said sensor assembly with said body; and
moving said sensor assembly with respect to at least one of said body and target area during said movement.

72. The method of claim 71 further comprising:
generating another movement of said body by said actuator member; and
moving said body with respect to said target area during said movement.

73. The method of claim 72 wherein said generating comprises one of:
moving said sensor assembly and body sequentially; and
moving said sensor assembly and body simultaneously.

74. A method for generating images of a target area of a physiological medium by an optical imaging system, said images representing two- or three-dimensional distribution of properties of hemoglobins in said target area, said method comprising the steps of:
positioning at least two wave sources and at least two wave detectors in a region of said target area substantially linearly along a straight line;
defining a scanning unit around said wave sources and detectors which has a scanning area which is smaller than said target area; and
generating at least one movement of said wave sources and wave detectors to move at least one of said wave sources and detectors to another region of said target area.

75. The method of claim 74 further comprising:
scanning said regions of said target area of said medium by irradiating said near-infrared electromagnetic waves thereinto and by generating output signals therefrom in response to said near-infrared electromagnetic waves detected by said wave detector.

76. The method of claim 75 further comprising:
repeating said scanning step at a plurality of regions of said target area, thereby enabling said optical imaging system to scan said regions having a total area which is substantially greater than said scanning area of said scanning unit and which is substantially identical to said target area.

77. The method of claim 76 further comprising:
terminating said repeating step after a pre-selected number of repetitions.

78. The method of claim 76 further comprising:
terminating said repeating step when said total area of said regions reaches a pre-selected portion of said target area.
